(19) United States
(12) Patent Application Publication
(10) Pub. No.: US 2011/0028825 A1
(43) Pub. Date: Feb. 3, 2011

(54) SYSTEMS AND METHODS FOR EFFICIENT IMAGING

(75) Inventors: Steven K. DOUGLAS, Danville, CA (US); Heinrich RODER, Steamboat Springs, CO (US); Maxim M. TSYPIN, Steamboat Springs, CO (US); Vishwas G. ABHYANKAR, Pittsford, NY (US); Stephen Riegel, Penfield, NY (US); James A. Schuster, Rochester, NY (US); Gene J. WOLFE, Pittsford, NY (US)

Correspondence Address: LEVINE BAGADE HAN LLP, 2400 GENG ROAD, SUITE 120, PALO ALTO, CA, 94303, US

(73) Assignee: DATAPHYSICS RESEARCH, INC., Danville, CA (US)

(21) Appl. No.: 12/793,468

(22) Filed: Jun. 3, 2010

Related U.S. Application Data
(63) Continuation of application No. PCT/US2008/013318, filed on Dec. 2, 2008.
(60) Provisional application No. 60/992,084, filed on Dec. 3, 2007.

(51) Int. Cl. A61B 5/05; G06K 9/00

(52) U.S. Cl. 600/407; 382/128; 345/419

(57) ABSTRACT

A system and methods for more efficient review, processing, analysis and diagnoses of medical imaging data is disclosed. The system and methods include automatically segmenting and labeling imaging data by anatomical feature or structure. Additional tools that can improve the efficiency of health care providers are also disclosed.

### CROSS-REFERENCE TO RELATED APPLICATIONS

This application is a continuation of PCT Application No. PCT/US2008/013318, filed Dec. 2, 2008, which claims the benefit of U.S. Provisional App. No. 60/992,084, filed Dec. 3, 2007, both of which are incorporated herein in their entireties.

### BACKGROUND OF THE INVENTION

1. Field of the Invention

The invention relates to the analysis, processing, viewing, and transport of medical and surgical imaging information.

2. Description of Related Art

Proliferation of noninvasive medical examination imaging (e.g., computed tomography (CT), magnetic resonance imaging (MRI), positron emission tomography (PET)) coupled with a shortage of accredited radiologists, especially in the U.S., has increased the value of radiologists' time. Increased resolution of imaging technology is also driving the quantity of information needing review by radiologists. Hence, the demand for radiologists' time is expected to increase further for the foreseeable future.

Typical imaging workflow includes numerous tasks performed by the radiologist that do not require specialized cognitive knowledge of the radiologist. Furthermore, existing tools used for analyzing and processing imaging data are “home grown” and not optimized to present the relevant clinical information to streamline the cognitive process of diagnosis and thus minimize radiologist time.

FIG. 1 illustrates a typical imaging analysis process flow, for example when used with a teleradiologist (i.e., a radiologist receiving the examination file for analysis over a computer network away from the examination site). The images are first acquired, for example with the CT or MRI machine. The compiled examination file of image data is then routed to clinical services.

Clinical Services is normally located at the image acquisition location. And are involved in the pre and post image acquisition process. They are responsible for generating the patient's exam file documentation, prepping the patient, coordinating image acquisition and post scan exam file organization. This may include image selection for Radiologist review. In addition to scanned images, patient files include documentation such as the Referring Physicians Report. These patient exam files are then made available to the Radiologist typically through a Picture Archive Systems Communications (PACS) approach. PACS refer to computers or networks dedicated to storage, retrieval, distribution and presentation for medical imagining. The most common PACS format is the Digital Imaging and Communications in Medicine (DICOM) format.

After clinical services, the examination file is then transmitted over a network to a teleradiology data center. The examination file undergoes quality assurance checking at the teleradiology data center. The examination file is then assigned to a radiologist and is placed in a queue at a teleradiology viewing location. When the (locally or remotely located) assigned radiologist is available to view the examination file, the radiologist then receives the entire examination file over a network (e.g., the whole file is “pushed” to the radiologist). The radiologist then examines and analyzes the examination file. The radiologist then creates a diagnosis report and sends (e.g., faxes) the report to the health care facility. Each of the steps in the typical imaging analysis occurs subsequent to the previous step.

Radiologists typically review image data using the “page” method or the “scrolling” or “cine” method. The page method is a legacy approach of simply paging through an exam file one image at a time. The page method is an inefficient artifact left over from the days of reading a few x-rays at a time. However, radiologists are now regularly required to review hundreds to thousands of two-dimensional images for a single patient. Using the page method this review is tedious and error-prone, and does not scale well to the large, and ever-increasing, number of images for each examination.

The scrolling method is where hundreds to thousands of images are stacked like a deck of about 100 to about 7,000 images. Using the scrolling method, the Radiologist scrolls up and down through the image slices several times developing a mental image of each organ in the image. The radiologist therefore performs a repetitive review of the same images merely to create the three-dimensional image in their mind. The scrolling method still lacks a three-dimensional image, can be time consuming, can be difficult even for trained radiologists to comprehend—and is especially difficult for a non-radiologist to understand—and does not include substantial longitudinal and volumetric quantitative analytical tools. In addition, the radiologist needs to compare and contrast with the previous imaging studies performed on the same patient.

FIG. 2 illustrates a typical series of two-dimensional radiological images that need to be reviewed by the radiologist, either using the page method or the scrolling method.

FIG. 3a illustrates a window for reviewing radiological images. The panel on the left shows a scout view 2 from the side of the viewed section of the body. The panel on the left shows the retrieved two-dimensional radiological image 4 for review. The highest and lowest images captured in the set of radiological images is shown by bracketing lines 6a and 6b.

As shown in FIG. 3b, the reviewing radiologist can select radiological images 4 to review by scrolling through the set of images, as illustrated by the selecting line 6c showing that the selected height of the radiological image 4 slice is between the bracketing lines 6a and 6b in the scout view 2.

FIGS. 4a and 4b illustrates that a single patient study or corpus can have more than one set of two-dimensional radiological images. For example, the study can have a set of unenhanced images and one or more sets of enhanced images. The enhanced images can be alternate images, such as images taken with the aid of an enhancement for example taken with a radiographic contrast agent in the patient. FIG. 4a illustrates that the menu 8 can be opened to select the enhanced (or unenhanced) image set. FIG. 4b illustrates an enhanced image 4 at a lower axial height than shown in FIG. 4a.

Because of the proliferation of medical imaging and the increased number of two-dimensional images for each examination, using existing methods radiologist are expected to shortly reach a point where a radiologist's daily work load becomes unsustainable.

In the current radiology workflow, the radiologist also usually performs many tasks that do not require his/her specialized knowledge. Theses tasks still consume valuable time from the main task of diagnosis. A systematic inclusion of radiology physician assistants (RPA) should occur in the clinical workflow with the additional responsibilities of patient assessments, separating normal from abnormal imaging exams, pathological observations, assembling and highlighting most relevant slices, informatics of current and prior studies for attending radiologist. There are indications that this kind of information and image staging are of significant value.

With the current systems, radiologists have to use keyboards, three-button mice (including scrolling wheel) and handheld dictation devices to interface with reading systems. These interface devices have served well for general computing tasks but they are not optimal for the specialized task of patient study interpretation.

Also, currently available systems can display the patient study (e.g., Radiology Information System (RIS) and PACS)) information, both images and informatics of current and prior studies over three separate monitors. A RIS stores, manages and distributes patient radiological data and imagery. The currently available systems follow predefined hanging protocols, but their static and rigid format for presentation and orientation of an imaging corpus can take a large amount of time. (The interpretation time varies from case to case. A normal exam for screening (e.g. yearly breast exam) takes just a few minutes but a differential diagnosis can take 10 to 15 minutes or more. A complex cancer case with prior studies can take an hour or more to complete the evaluation that follows RECIST (Response Evaluation Criteria In Solid Tumors) guidelines. Mostly these protocols have been designed in support of patient studies with a few two-dimensional images (e.g., x-ray film) and are inadequate and not easily scalable for the current and upcoming needs.

The expectation of what is useful in radiological clinical report varies depending on the referring physician's specialty. General practitioners (GPs) are very happy with a short text based report. Oncologists need to obtain information on the size, shape and temporal growth rate history of solid tumors and their related metastasis to assess patient prognosis and treatment response. They would like to obtain reliable measurements of specific abnormalities (lesions, calcifications), and have less interest in general exam information. On the other hand surgeons prefer very detailed analysis and 3D views of specific body part they are planning to treat, additional metrics and measurements are critical to the downstream medical care and procedures to take place. Their needs are different. Providing to them the relevant images and data not only helps them but it is also a very powerful marketing tool for radiology practices.

With high resolution imaging equipment, there are no preferred axial and planar directions. In most cases the images are taken as slices in the axial direction. But these slices are so thin and closely spaced that from this data one can project images in other two planes, front-to-back and side-to-side. The point (voxel) density is isotropic, and the image slices being displayed as axial, etc. is purely historical (in the sense that it is how the old X-ray film images were viewed) and has little practical value now.

Therefore, software and/or hardware tools to present this large and growing set of information that streamlines the cognitive process of diagnosis to optimize radiologists' time are desired and needed. Further, software and/or hardware tools to speed the qualitative and quantitative analysis of high-resolution imaging are desired. Further, software and/or hardware to facilitate the services of local or remote RPAs or SAs (specialist assistants), or software to assist in the tasks of the radiologist are desired. Moreover, software and/or hardware are desired to optimize the cooperation between RA and radiologist. Additionally, better macro and micro level interfaces and navigational controls over patient study information (both images and text informatics) are desired. A more intelligent, context-sensitive way of communicating, presenting and manipulating the relevant information that is in synch with radiologist's thinking process is also needed. This would free radiologist to concentrate on the task of image interpretation. A system that can assist in the creation of diagnostic reports specific to the audience of the report is also desired. There is also a need to display and analyze radiological images in a (historically) unbiased manner to obtain as much clinical information as possible.

### SUMMARY OF THE INVENTION

A system and method for more efficient medical and surgical imaging for diagnosis and therapy is disclosed. The data analysis process flow can be configured to have the radiologist perform review and analysis of the examination file concurrent with the examination file being transmitted to the teleradiology data center and the examination file quality assurance. Further, the teleradiologist can pull merely the desired part of the corpus and accompanying information from the examination file instead of receiving the entire examination file with the entire corpus. Functionally, the RA can perform his or her tasks on the data before the radiologist gets the data (i.e., but after the procedure has been performed). Physically, the RA can be co-located with radiologist or where the images are taken, at the teleradiology central service location, or elsewhere with a sufficient computer and network connection (e.g., located internationally).

The system can automatically assign anatomical labels to the corpus by comparing the corpus data with a reference database pre-labeled with anatomical information. The system can use haptic interface modalities to provide the health care provider with force feedback and/or three-space interaction with the system. The user can navigate (e.g., scroll) by organ or organ group or region/s of interest (e.g., instead of free three-dimensional location navigation), for example, where the critical pathology is easily viewable. The system can have various (e.g., software) navigation tools such as opacity control, layer-by-layer peeling, fly through, color, shading, contouring, remapping, addition or subtraction of region/s of interest, or combinations thereof. The three-dimensional navigation parameters can be used to specify three-dimensional hanging protocols and/or can be used in real time. The three-dimensional navigation view can be automatically synchronized with the multi-plane view and simultaneously displayed to radiologists. The organ selected can be shown more opaque (e.g., completely opaque), and the remaining organs can be shown less opaque (e.g., completely transparent, shadow form or outline form). The selected organ (and/or other organs) can be shown to the level of slices. (What we like to do is similar to the old novel/movie “fantastic voyage” but without shrinking people)

The system can have extender tools that can increase the efficiency of the interaction of the radiologist and assistants. The extender tools can “push” preliminary processing of information to the system itself and to the assistants, and away from the radiologist. The extender tools can improve navigation through the corpus data, for example allowing three-space movement through a virtual (e.g., three-dimensional) construction of the corpus data. The extender tools can also allow showing and hiding of selected anatomical features and structures.

The system can enable navigation through the visual presentation of the corpus through clinical terms and/or anatomical (i.e., three-dimensional spatial location) terms. Clinical proximity includes organs that are in direct mechanical, and/or electrical, and/or fluid communication with each other. Navigation by clinical proximity or anatomical proximity can be organ-to-organ navigation.

Where appropriate, the system can utilize context-based (e.g., image-based icons instead of text) presentation of information to speed communication with the user. These abstracting icons can provide graphical summary to radiologist so that in most cases they don't have to take time to open the whole folder to access and assess information.

The system can create one or more diagnostic report templates, filling in information garnered during use of the system by the practitioner and assistants. The system can create reports for referring or other physicians or reimbursers (e.g., insurance company). The system can create reports formatted based on the target audience of the report. Diagnostic snapshots captured by the radiologist during the analysis of the corpus data can be attached to these reports.

### BRIEF DESCRIPTION OF THE DRAWINGS

FIG. 1 illustrates a known variation for analysis data flow, not the invention.

FIG. 2 illustrates an exemplary view of a series of two-dimensional examination images, not the invention.

FIG. 3a illustrates an exemplary screen shot of a scout view and a radiological image.

FIG. 3b illustrates the screen shot from the image set of FIG. 3a at a different axial slice than shown in FIG. 3a.

FIG. 4a illustrates a method of changing the image of FIG. 3a to an enhanced image.

FIG. 4b illustrates a screen shot of a scout view and an enhanced radiological two-dimensional image.

FIG. 5 illustrates a variation of a method for analysis data flow.

FIG. 6 illustrates a variation of a method for construction and segmentation of the corpus data.

FIG. 7 illustrates a variation of displayed output data from a variation of the auto-segmentation method.

FIG. 8 illustrates a variation of a display screen shot from the system.

FIGS. 9a through 9c are screen shots of top, side perspective and front perspective views, respectively, of a three-dimensional segmented volume of a section of a torso cut-away at a first axial length.

FIGS. 10a and 10b are screen shots of top and side views, respectively, of a three-dimensional segmented volume of a section of the torso of FIG. 9a cut-away at a second axial length.

FIG. 10c is a screen shot of cross-section A-A of FIG. 10a.

FIG. 11a is a screen shot of side perspective of a section of a three-dimensional segmented volume of a section of the torso.

FIGS. 11b, 11c, 11e and 11f are screen shots of views of cross-sections B-B, C-C, D-D, E-E, respectively, of FIG. 11a.

FIG. 11d is a rotated view of FIG. 11c.

FIG. 12a is a screen shot of a window displaying the three-dimensional segmented volume of FIG. 11f with a segmentation transparency control.

FIGS. 12b through 12i are screen shot of the window of FIG. 12a with various rotations of the volume and segmentation transparency settings for the differently segmented tissues.

FIGS. 12j and 12k are screen shots of the window of FIG. 12i with the volume viewed with various Hounsfield settings.

FIGS. 13a through 13c are respectively sequential screen shots of navigation of the viewing position towards and into a three-dimensional segmented volume of a section of the torso.

FIGS. 14a through 14z are screen shots of methods of the physician extender functions and methods of using the same.

FIGS. 15a and 15b are screen shots of an exemplary automatically generated report.

FIGS. 16a and 16b are screen shots of an authorized variation of the report of FIGS. 15a and 15b.

FIGS. 17 and 18 are screen shots showing a variation of the log showing report authorization and delivery, respectively.

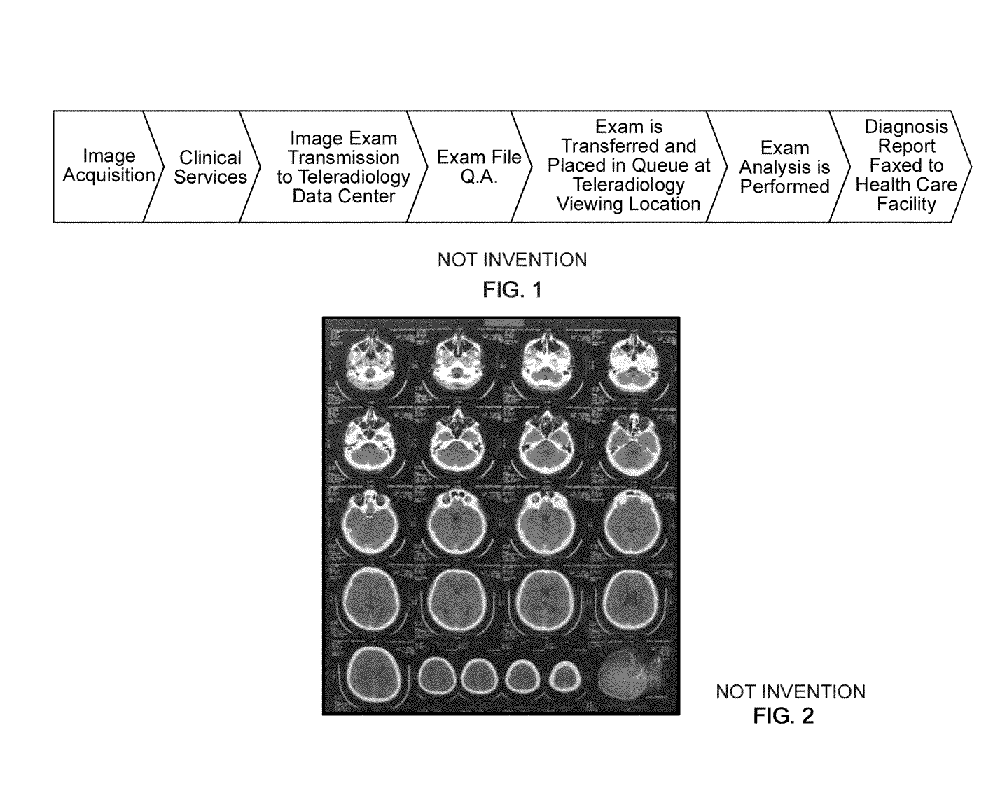

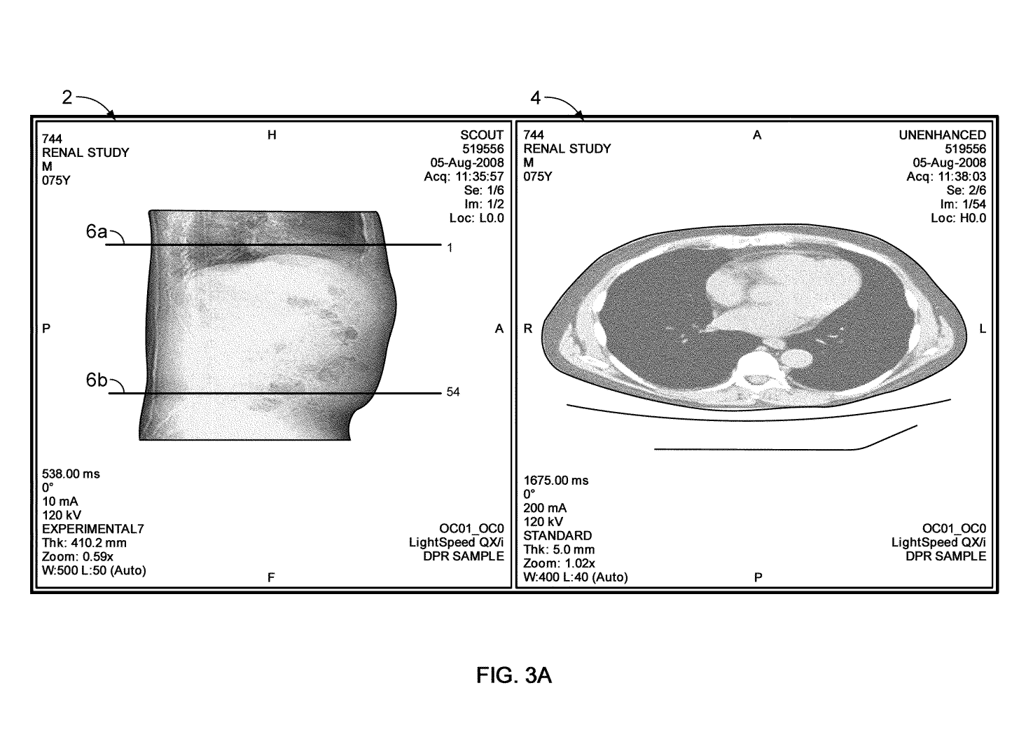

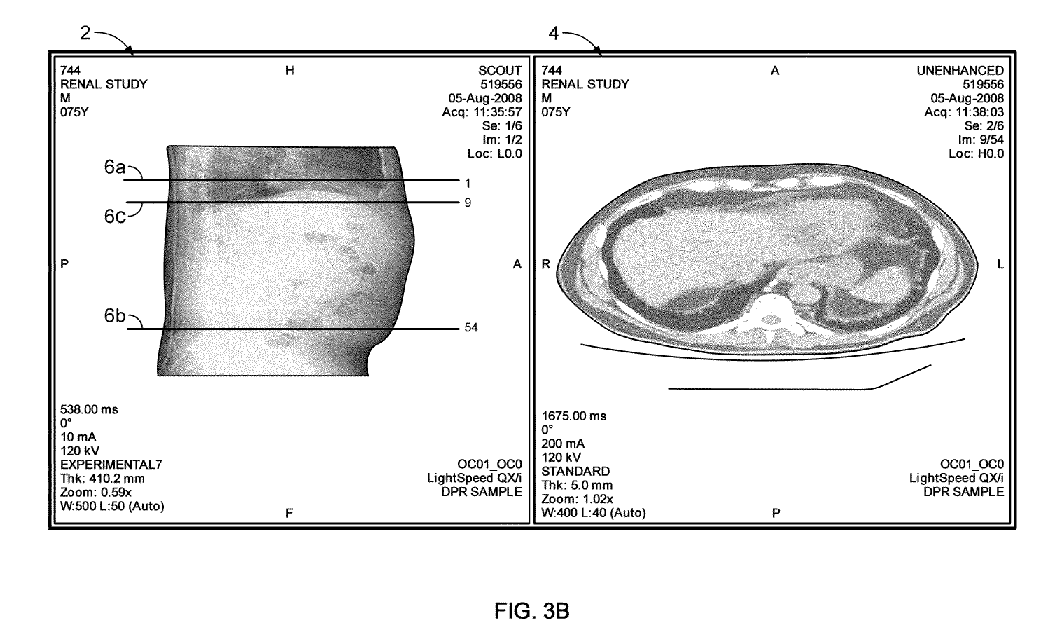

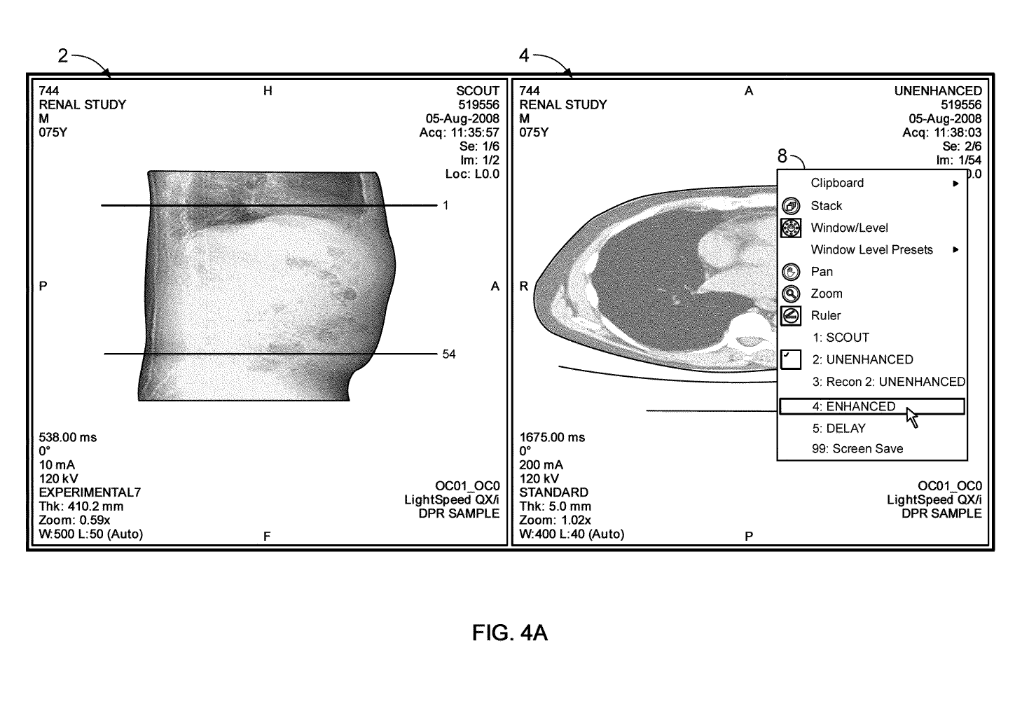

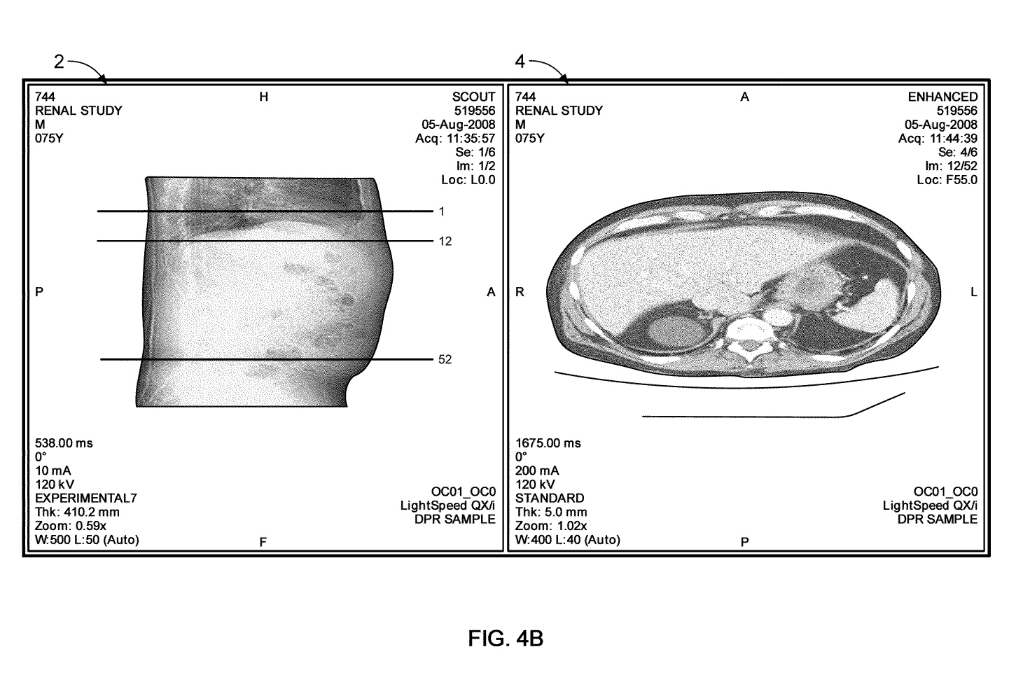

### DETAILED DESCRIPTION

The systems and methods disclosed herein can be used to process information (e.g., examination data files containing radiological data sets) for medical and/or surgical imagining techniques used for diagnosis and/or therapy. The systems can be computer hardware having one or more processors (e.g., microprocessors) and software executed on computer hardware having one or more processors and the methods can be performed thereon. The systems can have multiple computers in communication (e.g., networked), for example including a client computer and a server computer.

The examination data files can contain radiological data, modality information, ordering physicians notes, reason/s for the examination, and combinations thereof. The radiological data can include a corpus of data from the radiological examination and processing of the data from the radiological examination. The corpus can include data that represents images (e.g., PACS images, multiplanar images), objects, datasets, textual, enumerated fields, prior studies and reports and combinations thereof, associated with one or more actual physical locality or entity (e.g., tags that reflect some property in the real world).

### Concurrent Processing and File Pulling

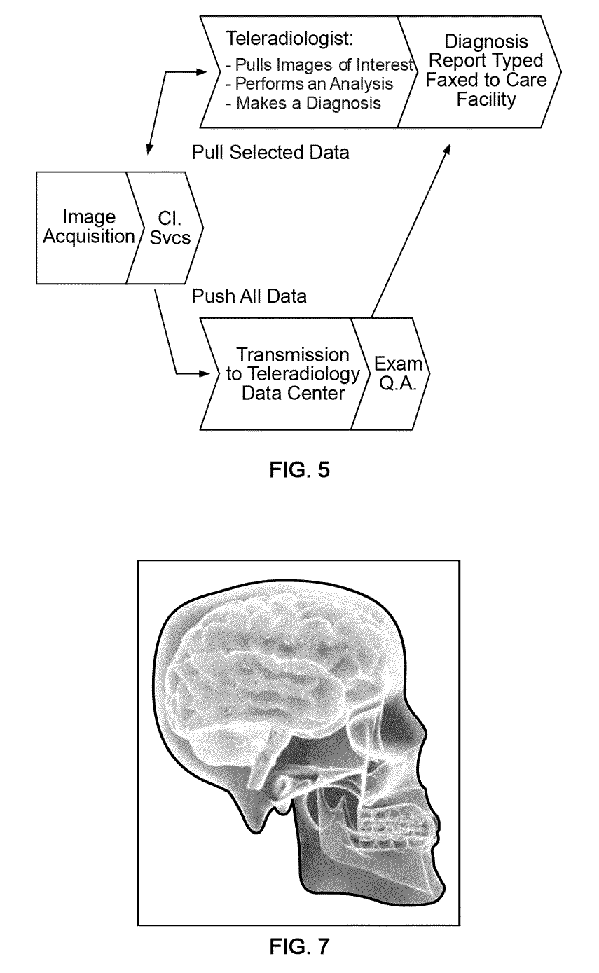

FIG. 5 illustrates that the process of radiological data analysis can begin with acquisition of the corpus, for example with the CT or MRI machine. The compiled examination file of radiological corpus data can then be routed to clinical services. Clinical services is described supra. Clinical services can augment the original captured data with relevant and valid findings, for example distilling, processing, extracting, augmenting, and combinations thereof, the imaging and/or patient information. Imaging information can include data representing actual images taken on a modality such as CT and/or MR. Imaging information can also include three-dimensional reconstruction in addition to the two-dimensional slice images. Imaging information can also include information about the imaging machine, contraction agents, or combinations thereof Patient information can include patient biography, demographic, habits and lifestyle (e.g., smoker, obesity, alcohol intake), the referring physician's order, prior conditions, notes taken during imaging, or combinations thereof.

The examination file can then be concurrently transmitted to a teleradiology data center, for example pushed as a whole file over a computer network. The examination file can undergo quality assurance checking at the teleradiology data center and/or viewing center and/or processed by a local or remote RPA.

When the assigned teleradiologist (or local radiologist) is available to view the examination file, the teleradiologist can then pull the desired corpus and the associated data for each portion (e.g., organ object/volume) of the pulled corpus over a computer network. The teleradiologist can then examine and analyze the pulled portions of the corpus of the examination file. If the radiologist desires additional corpus portions, the radiologist can then pull the additional corpus portions and associated data. If any data errors occurred during the transmission process, they can be corrected and sent to the assigned reading radiologist, for example, before the diagnosis report is generated.

Once the radiologist is satisfied by his or her analysis, the radiologist can then create an examination report, for example including a diagnosis. The radiologist can send (e.g., fax, e-mail, send via form in the system) the report to the health care facility or wherever else desired (e.g., teleradiology data center).

The teleradiologist can pull (i.e., request and transmit from over a network), analyze, and diagnose any or all of the corpus (e.g., the radiological data) at the radiologist's availability and/or concurrent with the examination file being pushed to the teleradiology data center and/or the examination file quality assurance. Queuing the examination file at a teleradiology data center awaiting an available radiologist is not necessarily required. The entire radiological data set need not be transmitted to the teleradiologist since the system can enable the radiologist to pull only portions of the corpus the radiologist wants to view.

### Corpus Construction: 3-D Construction and Auto-Segmentation

Corpus construction and segmentation functions and/or architecture in the software (e.g., executing on one or more processors) or hardware (e.g., a computer or network of computers having the software executing on one or more processors) system can process the examination file, for example, before the analysis by the radiologist and/or a pre-screen (or complete review) by a radiological technician or assistant. The corpus construction function or architecture can construct objects from the acquired radiological data.

The objects can be created by volumetric interpolation. The two-dimensional images and the associated data (e.g., attenuation) can be stacked and interpolation can be performed between the graphical information between the image planes to form voxels. The voxels can form one or more three-dimensional volumes. Each voxel can have interpolated data associated with the voxel. The voxels can be aliased

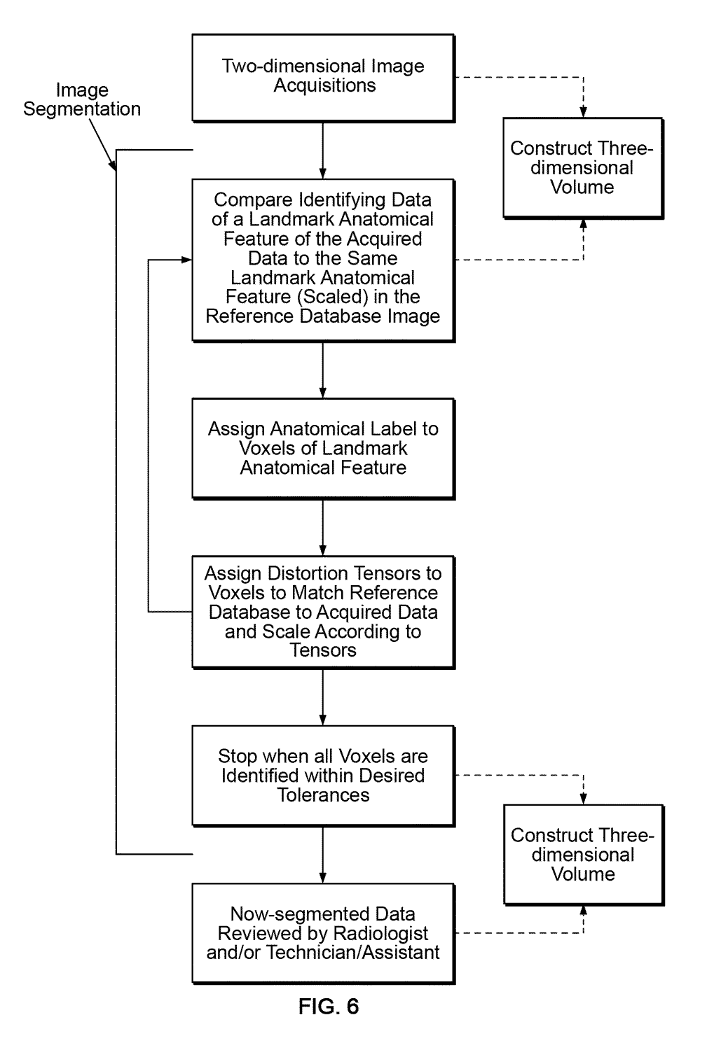

FIG. 6 illustrates that the corpus segmentation function or architecture can identify one or more (e.g., all) substantial anatomical features or structures in the corpus and link the label of the anatomical features or structures to the respective voxels (when three-dimensional corpus “volumes” are segmented). The anatomical features or structures can include specific organs, other tissue, cavities, pathologies and other anomalies, and combinations thereof.

The label of the anatomical feature can be illustrated during presentation of the data by a specific shading or color assigned to the voxel (e.g., bone can be white, liver can be dark brown, kidneys can be brown-red, arteries can be red, etc.). The shading can be opacity-based, using alpha blending, shadowing, smoking, VR (virtual reality) and visualization tools, and combinations thereof.

A reference database can be assembled from anatomical data from a different source. For example, constructed voxels based on the digital data from the Visible Human Project (e.g., Visible Human datasets) can be manually or computer assisted labeled with meta-data including a label for the particular anatomical feature or structure (e.g., pelvis, liver, etc.) of the respective voxel. Each voxel can contain data defining the location, anatomical label, color, Visual Human attenuation coefficient, and combinations thereof. Each voxel can be about 1 mm³. A single reference database can be used for numerous different patients' acquired imaging examination data.

Once the series of two-dimensional examination images are acquired, the corpus segmentation function and/or architecture can compare the anatomically labeled reference database data (e.g., in two dimensions or constructed or assembled into a three dimensional volume) to the acquired radiological data.

Each voxel of acquired data can be identified as being part of or not part of an automatically or manually selected data representing an anatomical feature or structure. This identification can occur by the software and/or hardware comparing at least one criterion (e.g., color and location) of each voxel of the acquired data to the criteria (e.g., color and location) in the voxel of the reference database. If the compared criteria (e.g., color and location) fall within a desired tolerance (e.g., +/−5%), then the acquired data can be tagged, labeled, or othenvise assigned with the anatomical label (e.g., pelvis, liver, femoral artery) of the respective reference database data.

The criteria of the anatomical features that can be compared criteria can include: contrast, attenuation, location (e.g., from an iteratively refined distortion field), topological criteria, connectivity (e.g., to similar adjacent anatomical features and structures in the examination data), morphology and shape descriptors (e.g., spheres versus rods versus plates), cross-correlation of attenuation coefficients, or combinations thereof.

The criteria can be refined and combined until the anatomical feature or structure is completely identified within tolerances (i.e. until there is no other anatomical feature or structure with a target score close to the assigned anatomical feature or structure). Each criteria can get a categorical score (i.e., fit, non-fit, ambiguous), which can be compared to check the quality of the assignment of the anatomical labeling/assignment.

Each time a complete or partial anatomical feature or structure (e.g., the pelvis, the liver, the femoral artery) is assigned in the acquired data, each voxel of the reference database data can be assigned a scaling or distortion tensor to scale (distort) the reference database according to the fit of the immediately previously assigned (and/or all other previously assigned) anatomical feature or structure. The scaling or distortion tensor can describe stretching (e.g., height vs. width vs. depth), rotations, shears and combinations thereof for each voxel. The reference database data can then be mapped to the acquired data using scaling or distortion tensors for the purposes of assigning anatomical labels.

The scaling or distortion field can be applied locally. For example, the amplitude of the scaling vectors can be reduced linearly, exponentially or completely (e.g., substantially to zero), as the location from the identified anatomical feature or structure increases. For example, the scaling or distortion field can be used to estimate only as accurately as necessary to obtain one confirmed seed within the next-segmented organ.

When iterating the acquired data by anatomical feature or structure (e.g., organ groups of the data base), for each identified anatomical feature or structure (e.g., organ) the distortion field can be updated to obtain better locations for the seeds (i.e., an initial voxel from which to compare for the desired anatomical feature or structure being segmented) of the next segmentation.

For example, after fully identifying, labeling and mapping the scaling or distorting tensors for the pelvis, the segmentation function and/or architecture can search at the approximate location of the liver for an attenuation coefficient that is similar from the reference database data (scaled/distorted for the pelvis) and the acquired data. Using a voxel corresponding in the acquired data to the liver in the reference database as a seed voxel, the voxels fitting within the tolerance of the corresponding organ (i.e., liver) can be labeled “liver” if the organ in the acquired data is similar in shape and attenuation to the corresponding organ (i.e., liver) of the reference database label. All voxels labeled as “liver” in the reference database data scaling or distortion field then get updated to match the “liver” in the acquired data.

If the corresponding organ is not identified at the seed voxel, or the resulting organ does not have a morphology (e.g., shape) or other criteria within the desired tolerances, the search can be restarted at another point of reference.

Although the scaling or distortion tensors are mapped for the reference database, supra, the acquired data could instead have scaling or distortion tensors mapped for the purposes of anatomical segmentation to map the acquired data to the reference database (as described, supra).

After mapping using the scaling or distorting tensors, the comparison process can be repeated using a new anatomical feature or structure. (E.g., the comparison can be performed organ group by organ group.) The anatomical features or structures can be assigned in order from easiest to identify (e.g., large bones, such as the pelvis) to hardest to identify (e.g., small vessels or membranes).

Voxels that can not be assigned an anatomical label can be labeled as “unassigned”. Anatomical features or structures that are not identified (e.g., because no acquired data sufficiently fits within the tolerances for the criteria for that anatomical feature or structure) can be noted. Unassigned voxels and noted unidentified anatomical features or structures can be brought to the attention of the radiologist and/or technician/assistant.

FIG. 7 illustrates an exemplary view of a three-dimensional segmented view of the sections of the brain with each section shown in a different color.

The segmentation function and/or architecture can provide for more efficient corpus data viewing, for example, because the anatomical features will already be labeled and can be distinguished by colors. Automatically identifying the anatomical features and structures also allows for better volume scalability (e.g., larger number of images can be more easily reviewed by radiologists and/or technicians/assistants, and larger number of examination files can be better processed by the system and method herein). The segmentation function and/or architecture also provides for more customizable analysis and use of more advanced analytic tools on the segmented data (e.g., processing of the data based on specific anatomical morphology: e.g., automatically identifying breaks in bones, tumors in organs).

When all the voxels in the acquired data are identified within a preset tolerance, the segmentation function and/or architecture can stop processing the acquired data. The now-segmented data can then be sent to a radiologist or technician/assistant for further review. The segmentation function and/or architecture and resulting three-dimensional data can be used in combination with page and scroll methods.

The resulting data can be navigated by organs, organ groups, region of interest or combinations thereof The resulting data can be navigated by clinical (i.e., anatomical) proximity and/or location (i.e., geometric physical) proximity. The resulting data can be transmitted through networks by organ, organ group, region of interest or combination thereof.

Mapping voxels to relevant medical information can aid the health care provider's decision making (e.g., diagnosis), for example. The mapping module can attach narrative and image medical reference material to each voxel or set of voxels (e.g., organ, organ group, region or interest, combinations thereof). The mapping module can be integrated with the segmentation module. The labels assigned to the voxels or set of voxels can be linked to additional information, patient-specific information (e.g., prior diagnoses for those voxels or set of voxels, or acquired data) or not (e.g., general medical or epidemiological information from one or more databases).

### Extender Tools

The system and method can include extender tools to facilitate preparation of segmented or non-segmented examination data, for example, by preparing the files by a physician extender (e.g., the radiologist or an RPA, imagining technologist/technician, or other technician or assistant before and/or during the final diagnosis) and to increase the efficiency of the review of the corpus and data for the final analysis and diagnosis. The extender tools can enable the physician extender and/or radiologist to be located remotely from the examination site physical and temporally. The extender tools may have a linked navigation module to lock (e.g., views of the same region of interest can be shown simultaneously and synchronously in both two-dimensional and three-dimensional view windows) together two-dimensional and three-dimensional views of the clinical information at diagnosis time. This module may implement a complex (e.g., the images can be presented that are based on the context of diagnostic interest and in a such a way that the relevant pathology is accentuated for rapid diagnosis by a radiologist or RA) set of logistical hanging protocols that determine the view and/or slice and/or orientation, and/or combinations thereof, that can, for example, be used by the clinician to diagnose. The extender tools can also improve the interaction and communication between the radiologist and the physician extender. The physician extender can highlight specific data for the radiologist and thus minimize the volume of examination data that radiologist would need to read before making diagnosis. The extender tools can provide specific protocol information and key corpus locations (e.g., organs) and findings to later stages of the diagnostic process, cross-references and correlation to previous relevant study data, compute qualitative and quantitative measurements, and combinations thereof.

The extender tools can optionally hide and show pre-existing conditions from the examination file. The pre-existing conditions can be represented visually in iconic form, as text, or as imaging information (e.g., snapshots). The physician extender can use the extender tool to highlight pre-existing conditions. The voxels (e.g., an entire or partial organ, area, organ group, etc.), can then be assigned a value as a pre-existing condition. The condition itself can also be entered into the database for the respective the voxels.

The physician extender can collect relevant information from the patient or file to indicate the disease state and supporting evidence for that disease state. The extender tools can enable to the physician extender to enter this information into the examination file and link all or part of the information with desired voxels (e.g., voxels can be individually selected, or an entire or partial organ, area, organ group, etc. can be selected). For example, attached information can include why was the exam was ordered and where symptoms are occurring, etc.

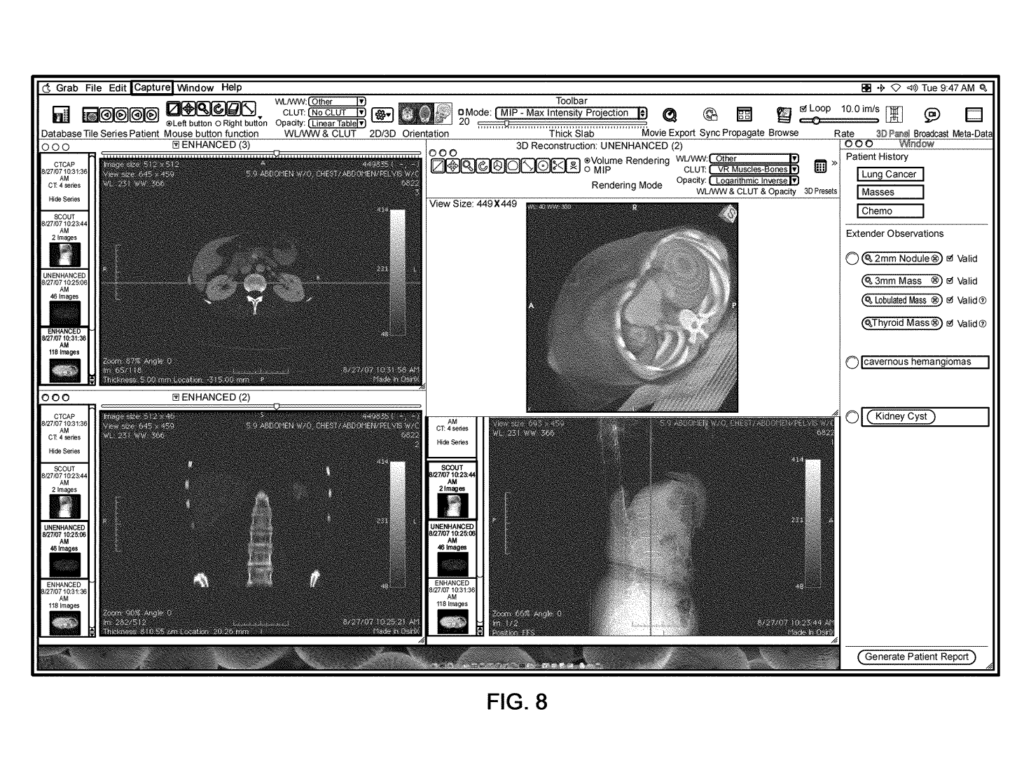

As shown in FIG. 8, a screenshot of the graphical user interface (GUI) of a variation of the extender tools, the extender tools can also provide navigation tools. The navigation tools can include synchronous three-dimensional (shown in the upper right quadrant) and two-dimensional navigation through one or more planes (e.g., shown in three planes). The three dimensional navigation can occur through sections of the corpus data, as shown.

The navigation tools can shown and hide selected voxels (e.g., voxels can be individually selected, or an entire or partial organ, area, organ group, etc. can be selected). For example, the user can select to show only the unknown voxels and the known pathological voxels (e.g., lung nodule, kidney stone, etc.) and associated organs. The user can then show and hide (e.g., invisible, shadow, only visible outline) surrounding anatomical features or structures, and/or navigate around and through the displayed volumes. Navigation parameters are described supra.

The selection of voxels to show and hide can be linked to text descriptions on the display. For example, the user can click the anatomical feature or structure (e.g., “lung nodule 1”, “liver”, “kidney stone”) to show or hide the same.

The extender tools can track, record and display metrics and performance benchmarks (e.g., time to review case, time for preparation of case by RPA, etc.).

The physician extender tool can have a collaboration module. The collaboration module can enable communication between a first computer (e.g., a computer of the diagnostic radiologist) and a second computer (e.g., a computer of a remote assistant), for example over a secure network, such as over the internet using a secure (e.g., encoded and/or encrypted) protocol. The collaboration module can transmit textual annotation and conversation, voice communication, and corpus series (e.g., organ) information (e.g., key frame, objects synchronized) communication between the first and second computers. The collaboration module can notify and call attention to either computer instantly of updated data, important findings, and questions requiring response from the user of the other computer.

The extender tools can be on multiple computers, for example on the workstation used for diagnostic reading and analysis. The extender tools can have a PACS/imagining tool, RIS tool, and combinations thereof, or be used in conjunction with existing PACS and/or RIS. PACS (Picture Archiving and Communication System) are computers, networks, and/or software dedicated to the storage, retrieval, distribution and presentation of the corpus. The PACS can show current corpus and prior case corpi. MS (radiology information system) include computers, networks and/or software that can show text file information, such as the case history, examination order and referring information.

The radiologist can have about one, two or three monitors (displays) (or fewer but larger monitors, for example). For example, two displays can show graphical imaging information, and one display can show textual meta information (e.g., case information, voxel and organ-specific information, such as for voxels and organs selected on the graphical displays). The extender tools can control the display of the graphical and/or text information. The extender tools can highlight specific textual information and key corpus locations.

The extender tools can display the segmented (or non-segmented) three-dimensional corpus alongside typical two-dimensional images, and/or the extender tools can show only the three-dimensional or only the two-dimensional images. For example, health care providers might be more comfortable adopting the system with the existing two-dimensional images in their existing format to use the existing knowledge to get a better and quicker feel for the three-dimensional (possibly segmented) images.

The extender tools can create and open DICOM standard file formats. DICOM file formats are generally universally compatible with imaging systems.

### Interface

Existing user interface devices, such as input devices (e.g., keyboards, one, two or three-button—or more—mouse with or without scroll wheels), can be used with the system and method. Additional or replacement interfaces can be used.

Other positioning devices that can be used include motion sensing, gesture recognition devices and/or wired or wireless three-space navigation devices (an example of the above includes location and motion recognition virtual reality gloves, or a stick-control, such as the existing three-space controller for the Nintendo Wii®), joysticks, touch screens (including multi-touch screens), or combinations thereof. Multiple distinct devices can be used for fine or gross control of and navigation through imaging data. The interface can have accelerometers, IR sensors and/or illuminators, one or more gyroscopes, one or more GPS sensors and/or transmitters, or combinations thereof. The interfaces can communicate with a base computer via wireless (e.g., Bluetooth, RF, microwave, IR) or wired communications.

Voice navigation can be used. For example, automatic speech recognition (ASR) and natural language processing (NLP) can be used for command and control of the study read process.

The interface can have a context-based keyboard, keypad, mouse, or other device. For example, the keys or buttons can be statically or dynamically (e.g., with a dynamic display, such as an LCD, on the button) with a programmable and/or context-based label, (e.g., an image of the liver on a button to show or hide the liver). The interface can be a (e.g., 10 button) keypad with images on each button. The images can change. For example, the images can be based on the modality (e.g., CT or MRI), pathology (e.g., cancer, orthopedics), anatomical location (e.g., torso, head, knee), patient, or combinations thereof, being reviewed.

The interface can include a haptic-based output interface supplying force feedback. The haptic interface can allow the user to control the extender tools and/or to feel and probe virtual tissue in the images. The voxels can have data associated with mechanical characteristics (e.g., density, water content, adjacent tissue characteristics) that can convert to force feedback levels expressed through the haptic interface. The haptic interface can be incorporated in an input system (e.g., joystick, virtual reality gloves).

The displays can be or incorporate three-dimensional (e.g., stereotactic) displays or display techniques.

The interface can include a sliding bar on a three-dimensional controller, for example, to de-noise images.

The interface can detect and incorporate brain activity of the radiologist or RPA and translate them into navigational commands and thus reduce or eliminate the need for keyboard and/or mouse interface. (e.g. See http://www.emotiv.com/)

### Context-Based Presentation

The system and method can communicate information using intelligent, context-sensitive methods for relevant information. For example, graphical icons (images) can be used instead of text for data folders and shortcuts (e.g., icons to indicate content of technical notes, referring specialist icon, folders on the hard drive).

The system can also provide (e.g., in the extender tools) automatic segmentation to bring forward most relevant part of organ or region of interest. Better measurement tools for volume, size and location etc.

The system can compare the current data for previous data for the same patient. The system can highlight the changes between the new and old data.

The system can generate a “key image” or keywords for data. For example, the system can cull important information and generates a single interface that shows the contextually relevant data to the radiologist while the radiologist reviews the images.

The system can automatically tag or highlight key images and meta data, for example when the image or data matches that in a key database. The tagged portions of the corpus and meta data can be shown first or kept open during the analysis of the corpus by the radiologist. The key database can be a default database with typical highlighted portions of the corpus and meta data. The radiologist can edit the key database for his/her preferences. The key database can be altered based on the patient history.

Icons used on the interface, and in the extender tools, and displayed elsewhere can be context-sensitive abstracted icons. The extender tools can compile data into folders and represent the folders on the display with abstract, context-sensitive folder icons. For example, the icons can represent details of various patient information folders. For example, the folder with data on the pain symptoms can be symbolically represented with the numerical pain level shown on folder (e.g., in the color representing the intensity of the pain from blue to red).

Iconic representation of common specific disease processes can be abstract representational or specific image representation. For example, the file of a diabetic may be shown by an icon of a sugar molecule. The file of an osteoporotic patient can be shown by an icon of a broken skeleton. The file of a hypertensive patient can be shown by an icon of a heart with an upward arrow. These examples, supra, are abstract representations.

Specific representations can have icons made using imaging data. A digital image of a wound can be scaled to the size of the icon (e.g., thumbnail) to form for the icon. A low resolution thumbnail of a bone break location can be used as an icon.

The icons and/or tagged or highlighted text or images can be linked to additional information (e.g., to whatever it is they represent). For example, the reason for the imaging can be shown on the case folder icon.

### Diagnostic Report Generation

The software can have a function and/or hardware can have architecture that can create a diagnostic radiology report template. The report template can be prefilled by the system with relevant information previously entered into the examination file and/or created by the system. The system can cull information from the acquired examination data for the report.

The function and/or architecture can automatically fill the report template based on observations produced during the exam. The system can partially or completely fill the report template using information recorded from the actions of the radiologist and physician extender during their use of the system. The system can generate reports, using context sensitive, structured templates.

The module can input the context and clinical conditions when proposing and generating text of the report. The module can produce structured reporting. The structured reporting can allow the user and/or generating system to follow a specific process to complete a report. The structured reporting can force the format and content of the report into a format defined in a report database based on the inputs. The context inputs can be based on the clinical conditions.

A limited number of case context specific questions can be answered by the radiologist. For example, the system can provide a bulleted list of options for variables within all or part of the report template for the radiologist to select from to partially or completely complete the report. The completed report can then be presented to the health care provider for review before filing the report.

Computer Aided Detection Diagnosis (CAD), Computer Aided Radiography (CAR), or other additional algorithmic inputs to the system could be used to increase efficiency. A CAD module can use diagnostic data generated by a diagnostic algorithm and incorporate the diagnostic data into the physician extender dataset presented at diagnosis. The CAD module can produce a diagnostic result (e.g., “there is an anomaly at [X] and [Y] locations that the health care provider should investigate”). A CAR module can produce a location of interest (e.g., but does not generate a clinical interpretation or finding) (e.g., “you should investigate at [X] and [Y] locations”).

The system can have a microphone. The user can speak report information into the microphone. The system can use automatic speech recognition (ASR) and natural language processing (NLP) to process the speech and assemble the report.

The report can have fixed fields (e.g., may vary from report to report, but usually selected by the system and usually not changed by the physician) and variable fields (e.g., usually filled in by the physician with little or not assistance from the report generation software or architecture). The reports can be searched within the variable fields or across the entire report (i.e., fixed and variable fields).

Inputs from the referring physician, nurse, etc. can all be entered automatically and/or remotely (e.g., even by the referring physician) into the diagnostic report. For example, at old injuries or histories can be entered into or (hyper-) linked to the report.

Once approved by the health care provider, the report can be automatically transmitted by the system, in an encrypted or non-encrypted format, to the desired locations (e.g., radiologist's file, patient file elsewhere, referring physician's file, teleradiology data center, insurance reporting computer, etc.)

A report can, for example, follow a four section structure, or any combination of these four sections: (1) demographics; (2) history; (3) body; (4) conclusion. The demographics section can include the name, age, address, referring doctor, and combinations thereof. The history section can include relevant preexisting conditions and a reason for the exam. The body section can include all clinical findings of the exam. The conclusion section can have staging (e.g., the current disease state and progression of a clinical process) information and clinical disease processes definitions and explanations.

### Regulatory Compliance

The system can capture and automate compliance and regulatory data. The software can have a function and/or hardware can have architecture that can perform corpus chain quality control and calibration for the examination corpus data. The system can automate data collection, tracking, storage and transmission for quality, reimbursement, and performance purposes, for example.

Radiologist performance, such as retake tracking, technical competencies and ancillary training, patient satisfaction, time per case, quality improvement (QI) feedback, etc. can be stored, tracked, and sent to the radiologist, hospital, medical partnership, insurance or reimbursement computer, or combinations thereof. Policy management and pay for performance data can also be stored and tracked.

The system can have a database with regulatory and/or compliance information. The system can have a module to generate the reports and certificates necessary to demonstrate compliance with the regulatory and/or reimbursement and/or other administrative requirements.

Peer review can also be requested by the software. A peer review process module can include the physician extender and segmentation extensions to the corpus for the purpose of sharing a read and interpretation process. The module can share all or part of the system functions with one, two or many other health care providers (e.g., RAs, RPAs, doctors, technicians), for example, to collaborate (e.g., potentially gain a group consensus, pose a difficult condition to seek resolution experience) with health care providers at other locations (e.g., computers on the network). The peer review process module can be initiated as a result of direct user input to the system. The peer review module can be used synchronously or asynchronously. Synchronous use can be when a user starts an immediate peer consultation. Asynchronous use can be when the user requests that a peer consultation be held on a particular case at any time, and/or with a deadline.

The system can aggregate and file examinations. For example, the system can maintain a large scale database for examination aggregation for teleradiology centers. The system can provide specialization documents and file types for specific body regions and diagnoses.

The system can have a workflow module that can route examination files to the appropriate work queue. The workflow/ module can use a clinical interpretation developed by the extender and added to the examination file. The workflow module can use the clinical interpretation to determine the placement in the queue (e.g., based on urgency) and to which radiologist (e.g., based on how each radiologist's performance matches with the clinical interpretation) for final analysis, approval and signature. For example, a data center may have examination data files for 50 different types of procedures, and have two radiologists to read all 50 cases. The workflow module can route each examination data file to the relevant expert (between the two radiologists) for the specific examination data file.

The system can have a data interface to the practice management system. The system can send and receive data (e.g., be networked with) the HIS (health information system), RIS (radiology information system), PMS (Practice Management System), or combinations thereof.

The system can automatically send reimbursement information (e.g., over a network) to a reimburser's computer required for reimbursement. The system can automate pay per performance rules in a regulated business environment. The reimbursement information can include patient and examination information including what practitioners viewed what information at what time.

### Report Generation for Referring Specialist

The software can have a function and/or the hardware can have architecture that can create variations of (e.g., two different) final reports for the same study. For example, one report can be for the radiologist, one report can be for a surgeon, and one report can be for a family practitioner. The system can differentiate the reports, for example, based on the recipient (e.g., type of doctor). For example, the system can create a report with a first group of information for a surgeon and a second group of information for a family practitioner. (The surgeon may request more information particular to the morphology of the disorder, including portions of the corpus in the report. The family practitioner may request merely the conclusion of the report.)

The system can provide mechanisms to inform and prompt the radiologist to the need for additional metrics and measurements requested by the specialist. For example, the specialist can communicate over a network (e.g., on a secure website) to the system and request particular information.

The system can use delivery mechanisms (e.g., fax, e-mail, print paper copy) and report preferences defined by specialist class (e.g., orthopedic surgeon, family practitioner, insurance company) and then by individual specialists (e.g., Dr. Jones). The system can use context-based key portions of the corpus for the recipient of the report.

### General Data Report Creation: Business and Legal Reports

The system and methods can include software functions and/or hardware architecture to collect information file (automatically) to provide requested evidence. For example, when information retrieval is requested (e.g., for the discovery process for a legal case, such as for a malpractice or other law suit, or for business analysis and consulting), the functions and/or architecture can provide a checklist of desired data to select and deselect specific data, and automatically retrieve the information and produce reports. This functionality can save time for data retrieval during evidence retrieval/discovery purposes or for consulting purposes.

Examples of data automatically collected include: logs of who worked with the examination file data and when, who saw the information and when, who reported on the case and when, all dates and times of file access, changes and deletions, permission levels and the authorizing agency, the agents of the system, network communications to and from the system, and combinations thereof.

### Malpractice Safeguards

A legal check-list can also be provided and/or mandated during the analysis and diagnosis of examination files, for example, to protect the user against The system and/or method can also automatically perform steps to protect against legal liability. For example, the system can be configured to record unused or archived data in a read-only (i.e., non-editable, non-deletable) format to preserve the integrity of the data. For example, the system can be configured to only allow augmentation of the examination files (e.g., not editing or deleting of existing data). The dates, times, and users of all augmentations can be recorded. This can reduce malpractice incidents and insurance premiums.

### Exemplary Screen Shots

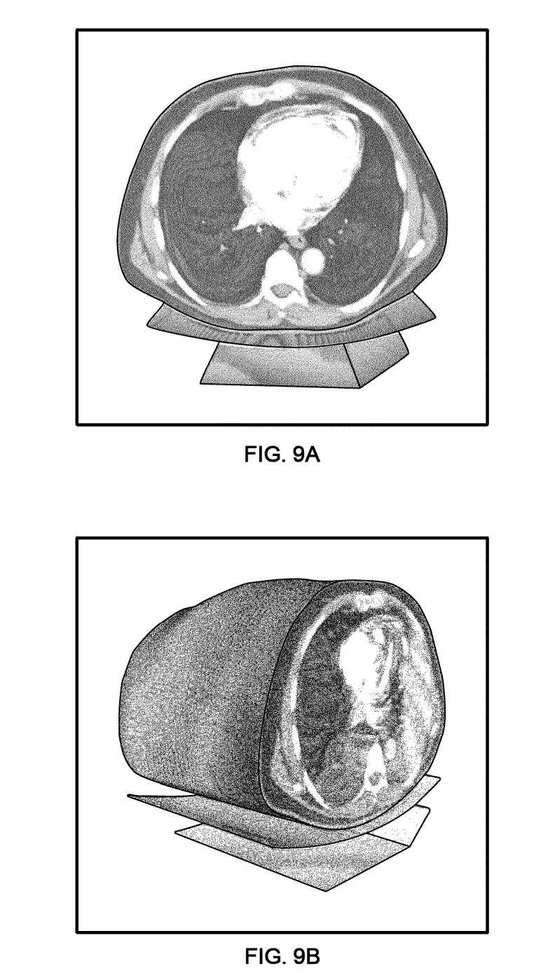

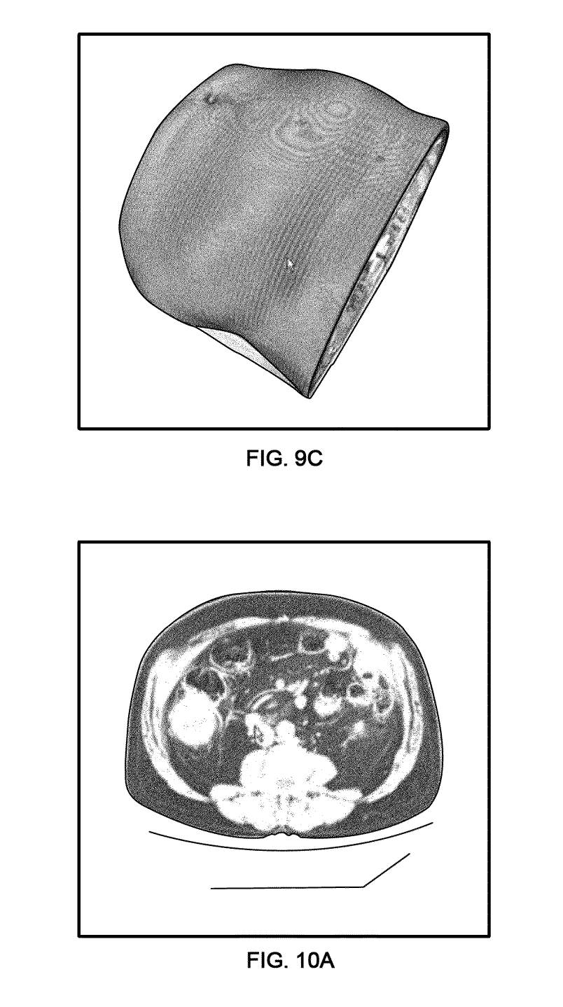

FIGS. 9a through 18 illustrate a variation of the system and method disclosed herein through screen shots captured during use of the system. The screen shots are non-limiting and merely shown to further describe the disclosed system and method.

FIG. 9a illustrates a three-dimensional volumetric composite of the captured data sets, as described supra. For example, the data set can be MRI or CT data for a length of a torso. FIG. 9b illustrates that the volumetric view of the corpus of the data can be rotated. FIG. 9c illustrates that the volumetric view can be further rotated, for example showing topological features of the skin, such as the navel (as shown), and/or features of the clothes, such as buttons (as shown).

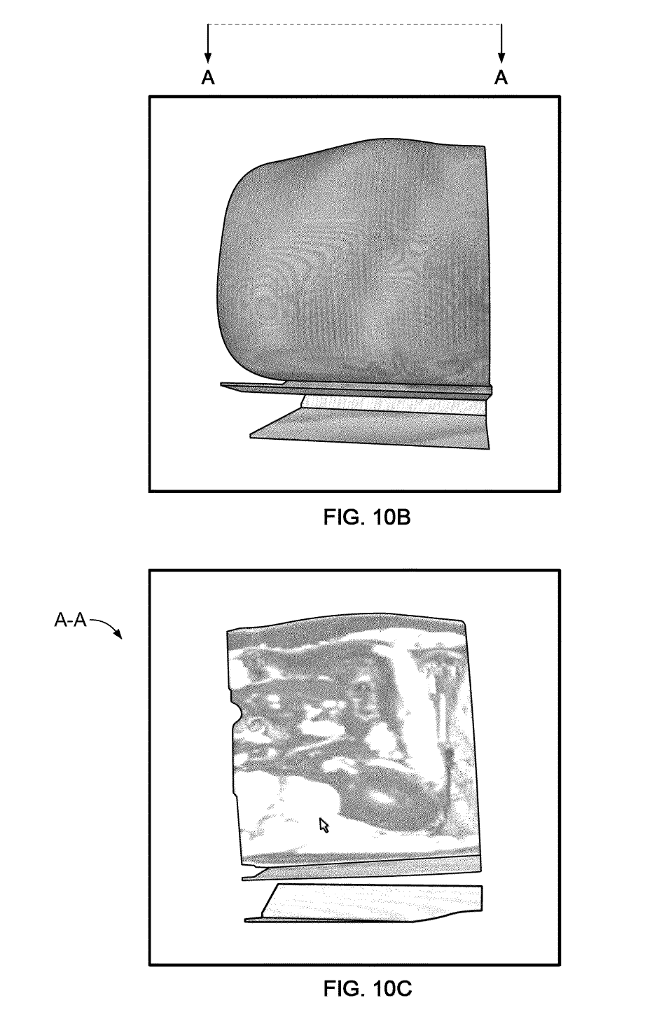

FIGS. 10a and 10b illustrates that the cut-away section can be moved along the axial length of the volumetric section (e.g., with respect to where the cut-away is shown in FIGS. 9a through 9c ). For example, the window or panel displaying the volumetric data can be used in conjunction with a scout view, as described supra, to control the depth of the cross-sectional plane in the volume dynamically (e.g., as the volume is being rotated and otherwise manipulated).

FIGS. 9a through 10a illustrate that the volume can be sectioned along a plane substantially perpendicular with a longitudinal axis. FIGS. 10b and 10c illustrate that the volume can have multiple sections and/or can be sectioned along the sagittal plane, or other planes such as the coronal, transverse or other planes or no-straight (e.g., curved, angled) surfaces.

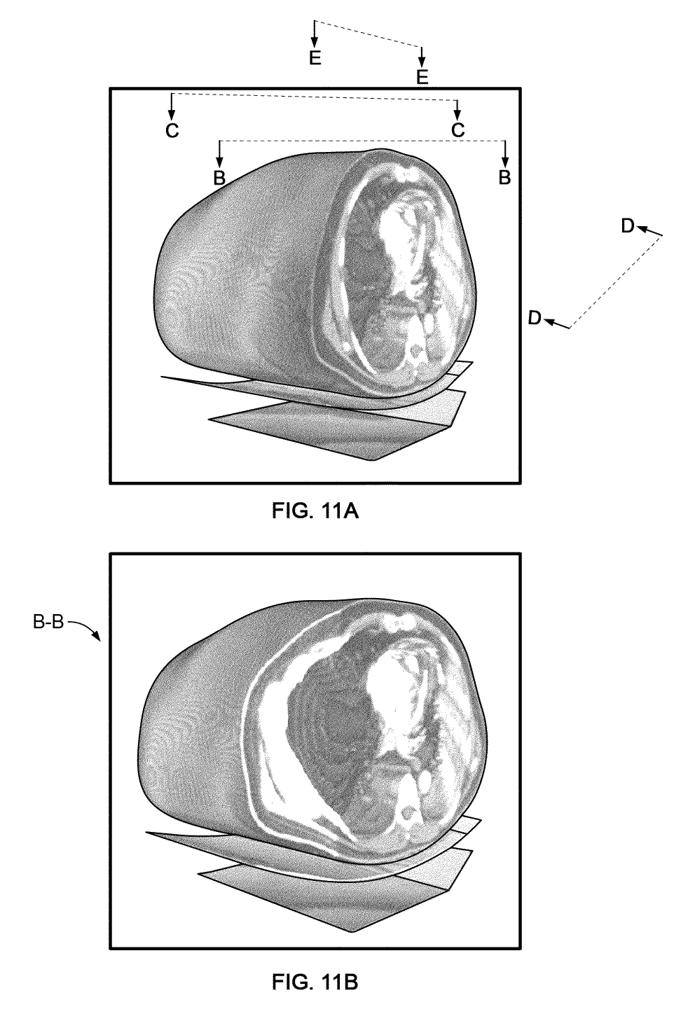

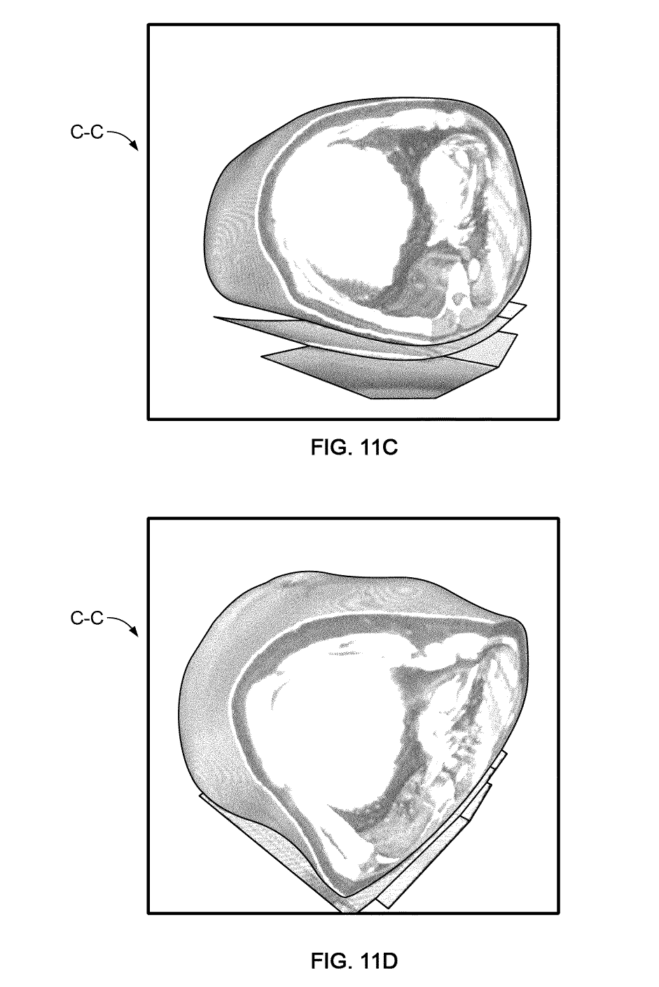

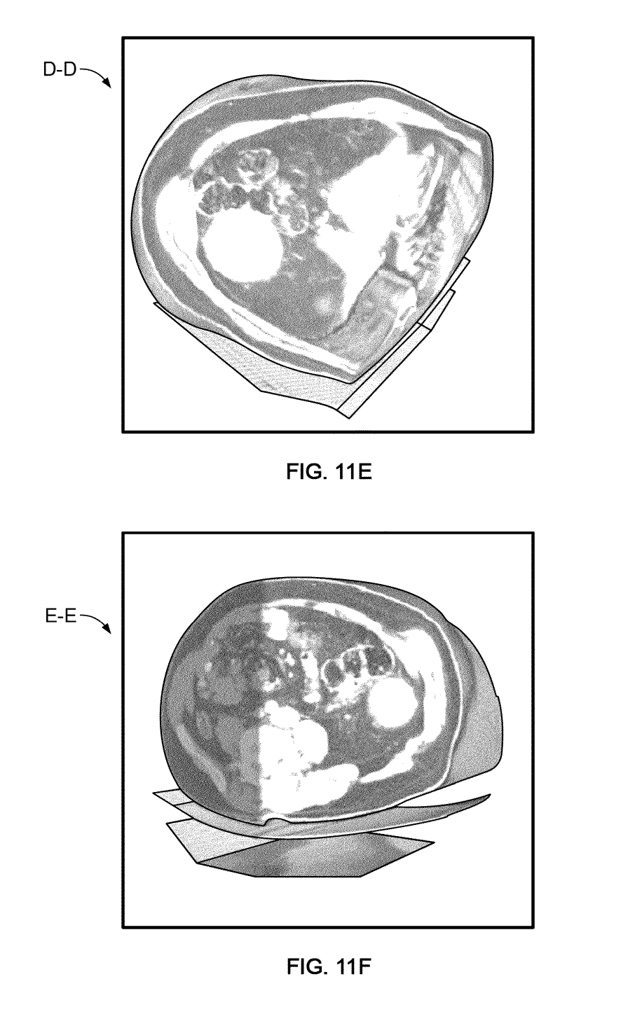

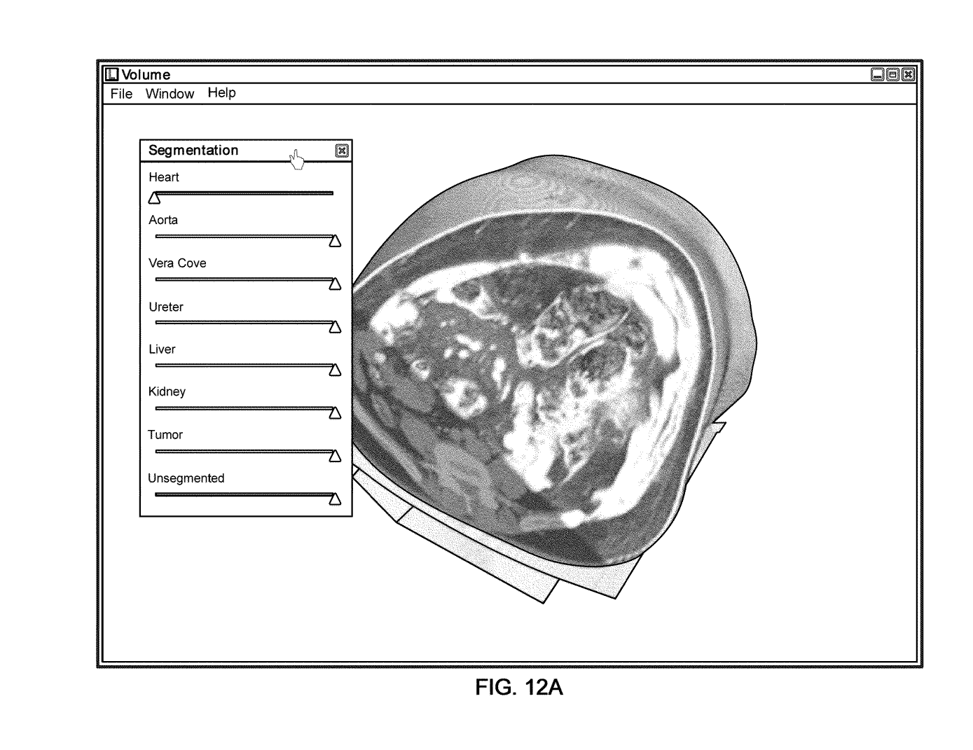

FIG. 11a illustrates that the volume can be sectioned, reconstructed and resectioned along a different sectioning plane or other sectioning surface. FIG. 11b illustrates a non-Cartesian sectional plane B-B (e.g., a plane not parallel or at a right angle to the sagittal, transverse or coronal planes).

FIGS. 11c and 11d illustrate a steeper-angled section of the volume that that shown in FIG. 11b. FIGS. 11e and 11f illustrate other sectional views of the same volume. The volume can be sectioned to reveal specific organs or other tissue.

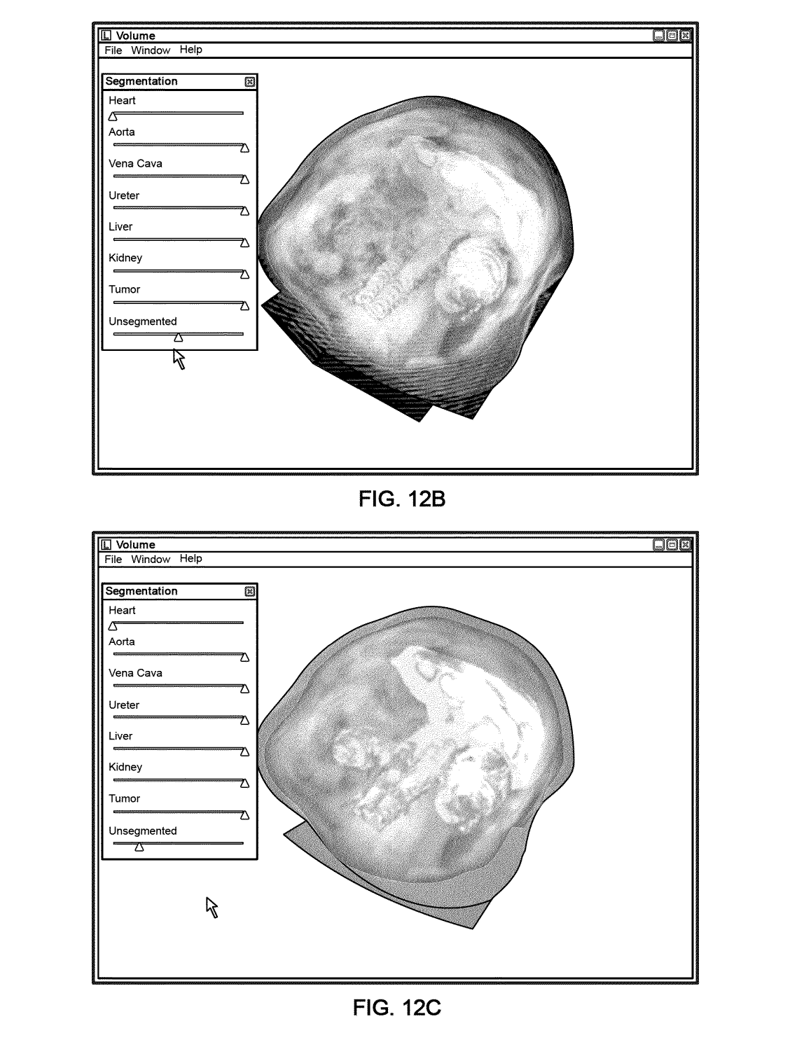

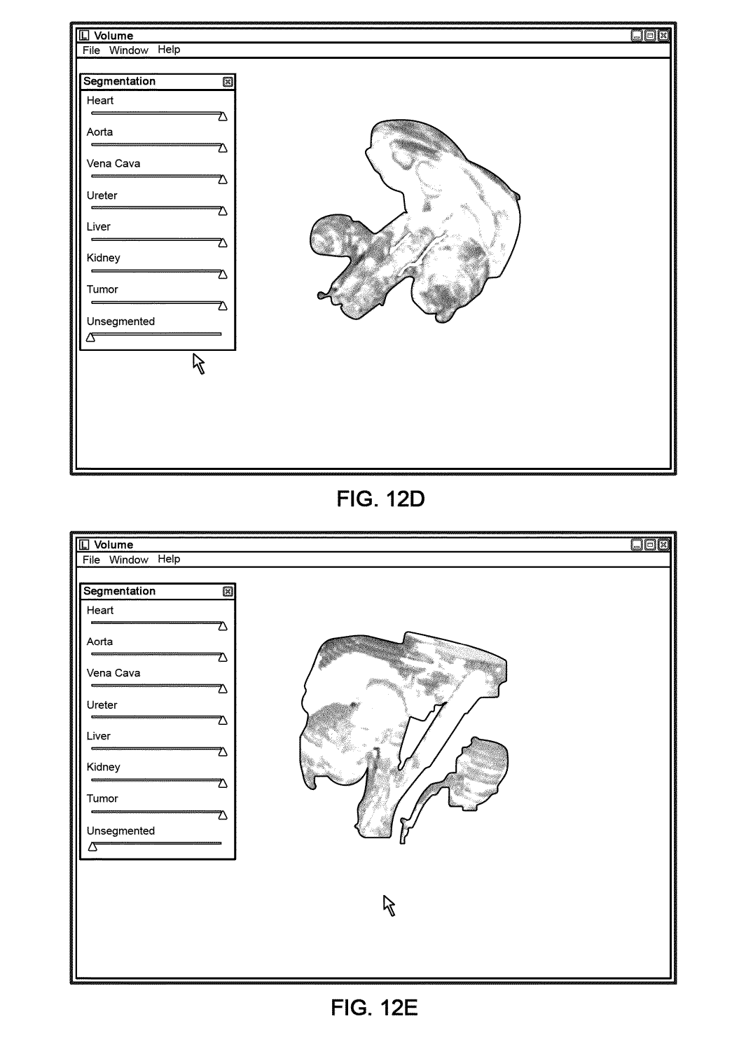

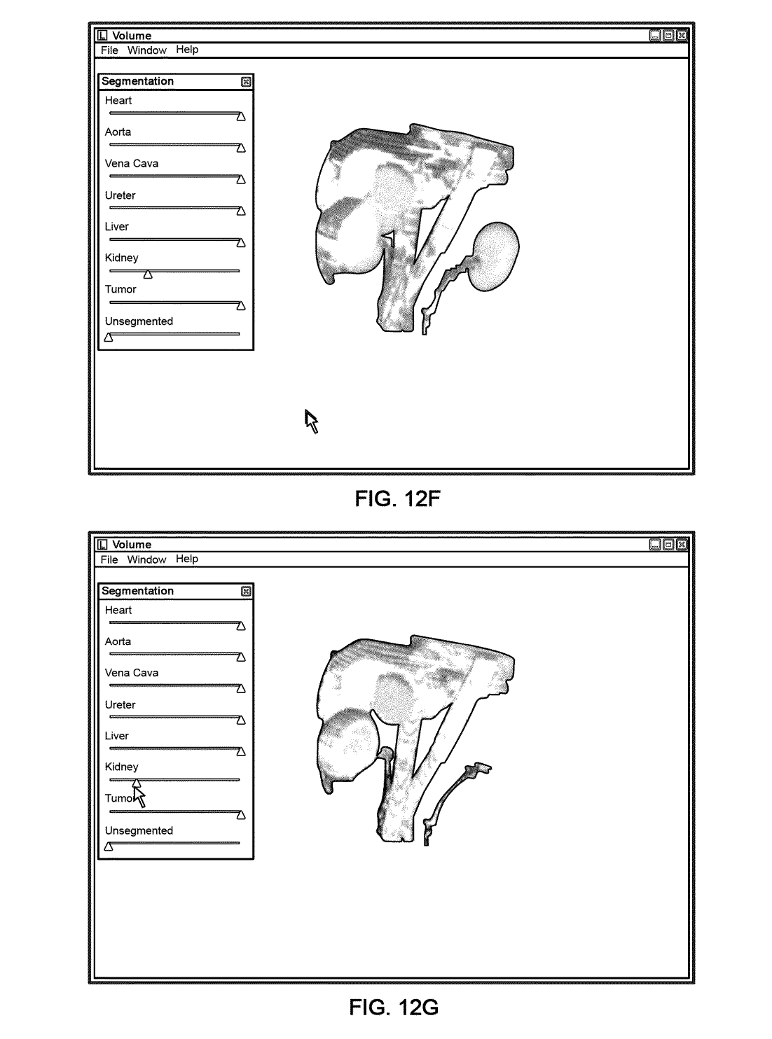

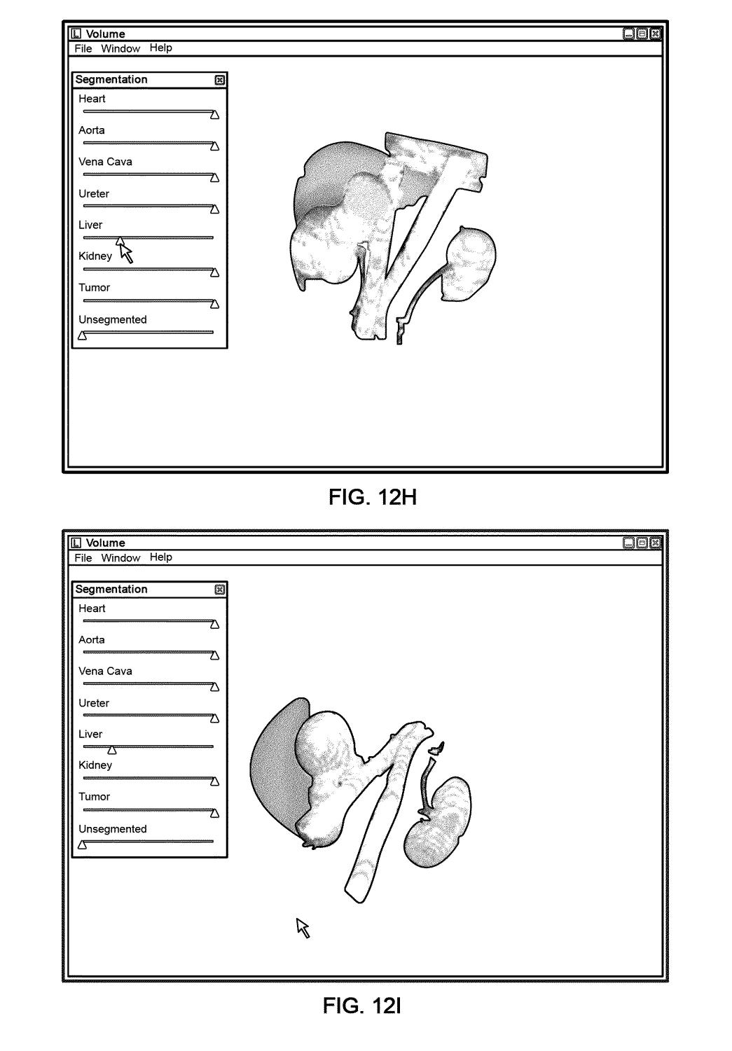

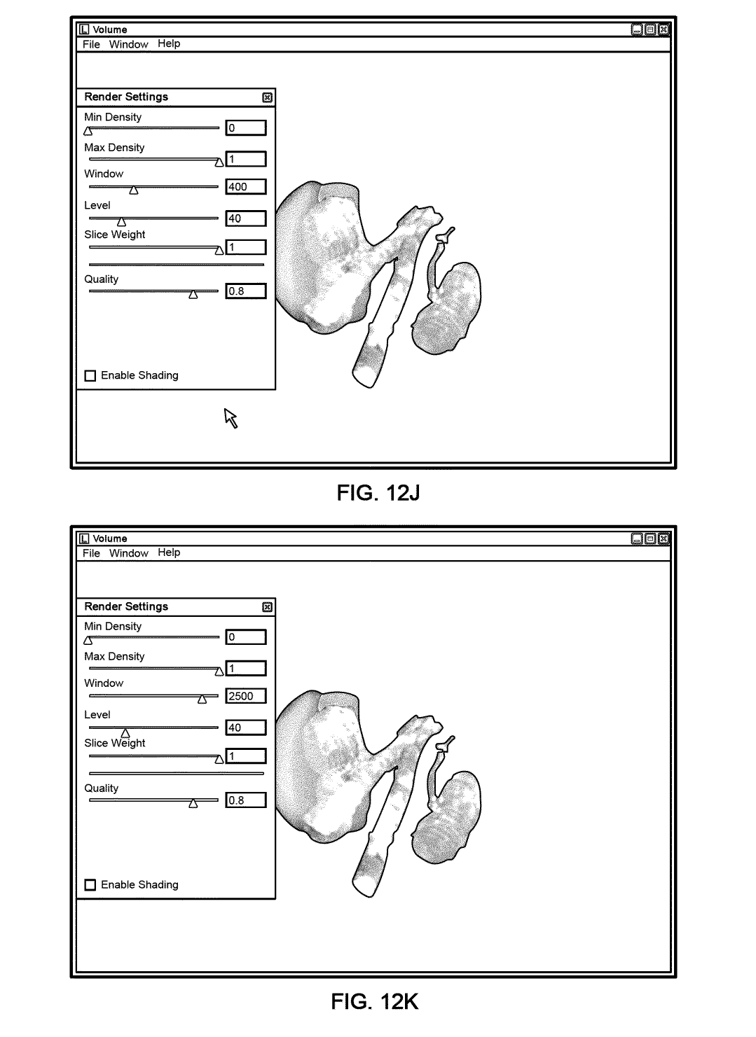

FIG. 12 illustrates that the system can produce a menu to control the transparency of individually segmented portions (e.g., organs, tissue, pathologies, or non-segmented data). The menu can have controls to receive numerical data, slides (as shown), other toggles, or combinations thereof. Each segmented portion can be individually controlled or controlled as a sub-group or group. Exemplary segmented portions shown in FIG. 12a include the heart, aorta, vena cava, ureter, liver, kidney, tumor and unsegmented data (e.g., volumes that did not fall within the data brackets required to qualify as a defined segmentation portion, for example this can include skeletal muscle, clothes, etc.). FIG. 12a illustrates that the heart volume can be set to be completely transparent (e.g., the slider is pushed all the way to the left) while the other segmented portions can be fully opaque (e.g., the sliders are pushed all the way to the right).

FIG. 12b illustrates that the unsegmented volumes can be made about 50% transparent, as shown by the position of the unsegmented slider control and the image transparency. FIG. 12c illustrates that the unsegmented volumes can be made about 75% transparent. FIG. 12d illustrates that the unsegmented volumes can be made about 100% transparent.

FIG. 12e illustrates that the volume can be rotated, scaled, translated, or combinations thereof whether any segmented data is partially visible or not.

FIG. 12f illustrates that the kidneys can be made about 70% transparent. FIG. 12g illustrates that the kidneys can be made about 85% transparent.

FIG. 12h illustrates that the liver can be made about 70% transparent. FIG. 12i illustrates that the liver can be made about 80% transparent and that the volume can be rotated.

Any of the segmented groups can be placed in any combination of states of transparency with respect to any of the other segmented groups, and/or limitations can be set for corresponding groups (e.g., the heart and blood vessels can be forced together or within about 20% of transparency of each other).

FIGS. 12j and 12k illustrate that the Hounsfield levels can be adjusted independent of (or linked to, but not shown) the transparency levels. For example, FIG. 12j illustrates a Hounsfield level of 40 and a Hounsfield window of 400. FIG. 12k illustrates a Hounsfield level of 40 and a Hounsfield window of 2500.

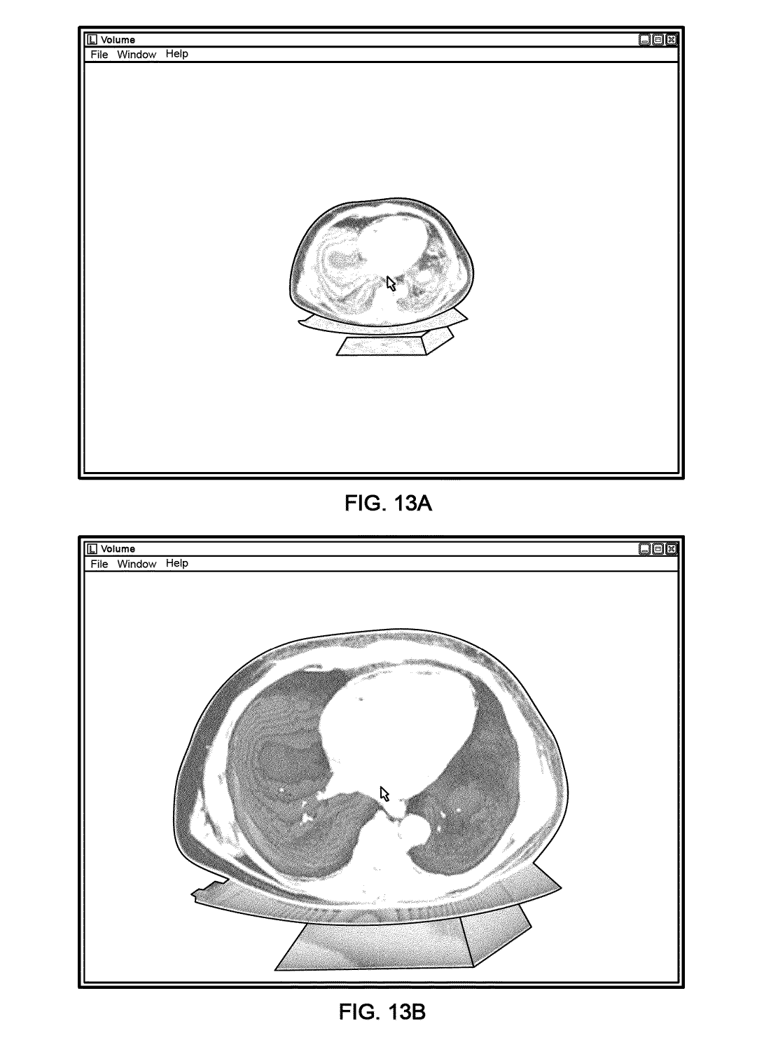

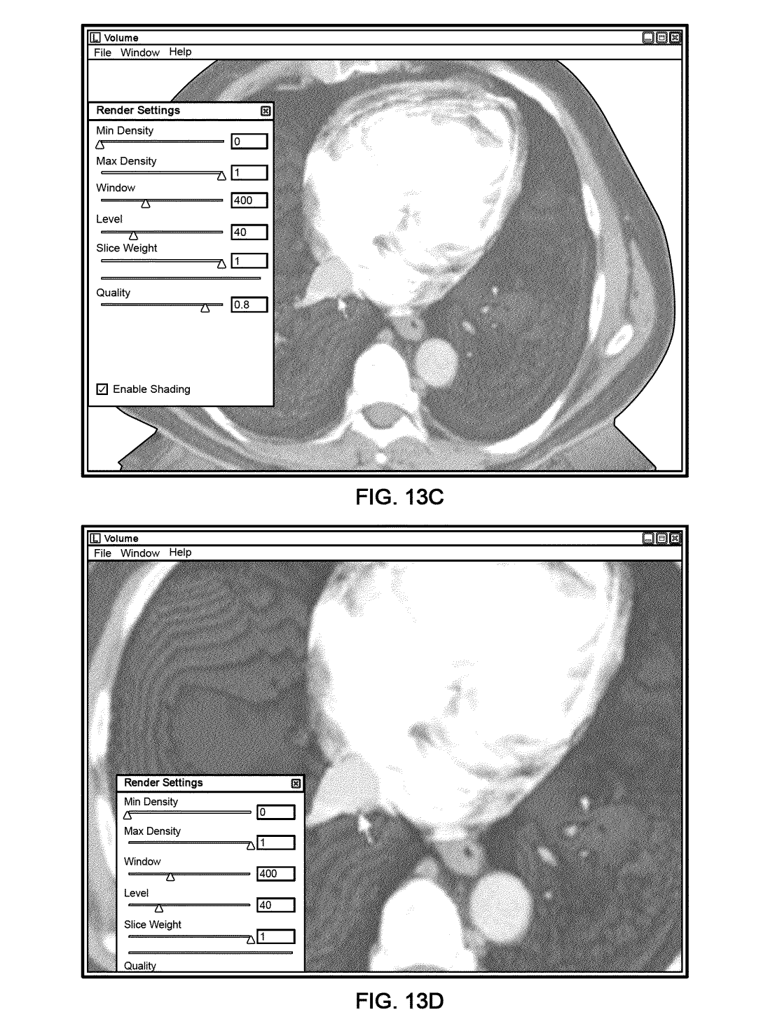

FIGS. 13a through 13d illustrate a sequence of views of translating and rotating the viewpoint relative to the volume. The viewpoint can be translated into the volume, as shown in FIG. 13d. A diagnostician can navigate or “teleport” inside of the volume to find a perspective most accommodating to investigate any desired aspect of the image.

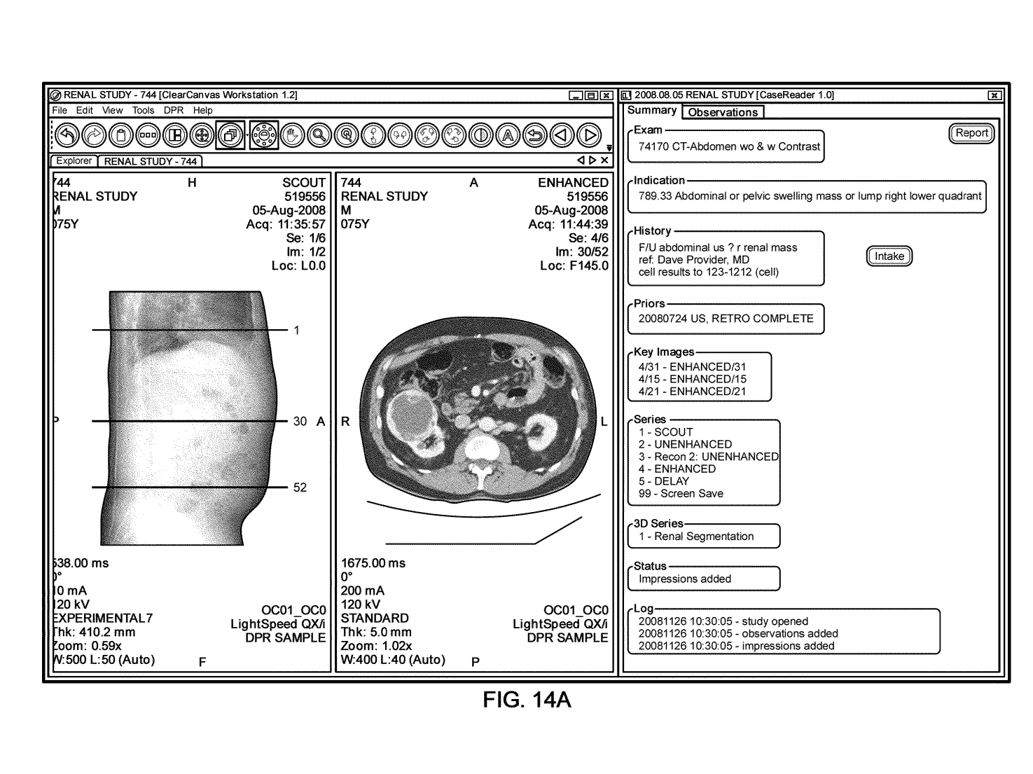

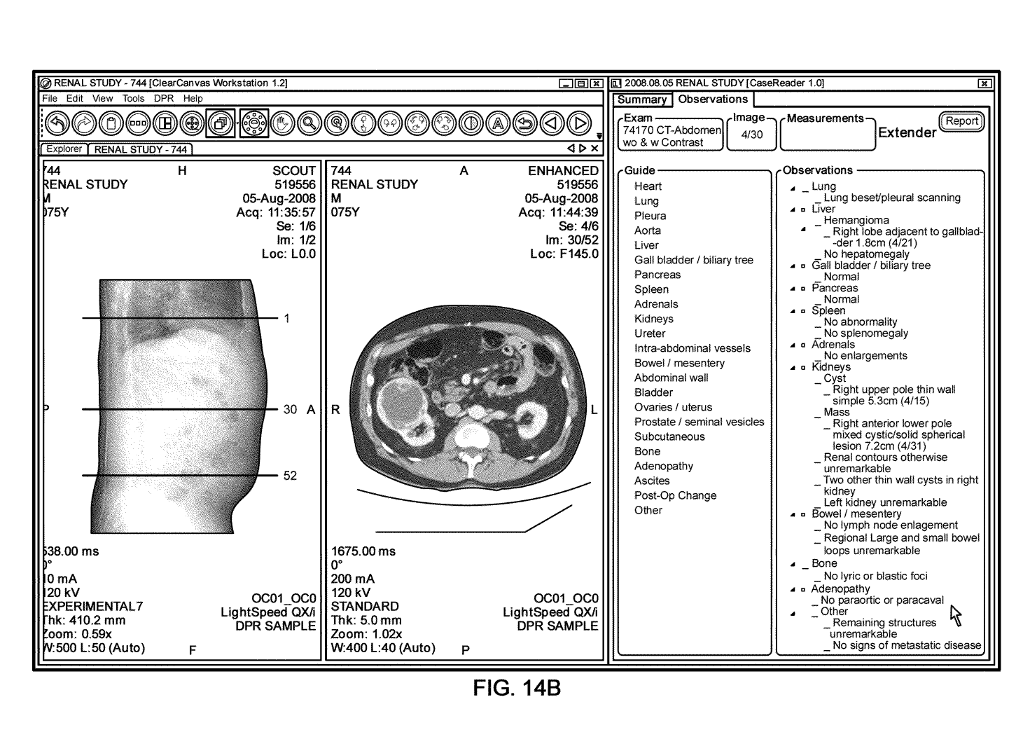

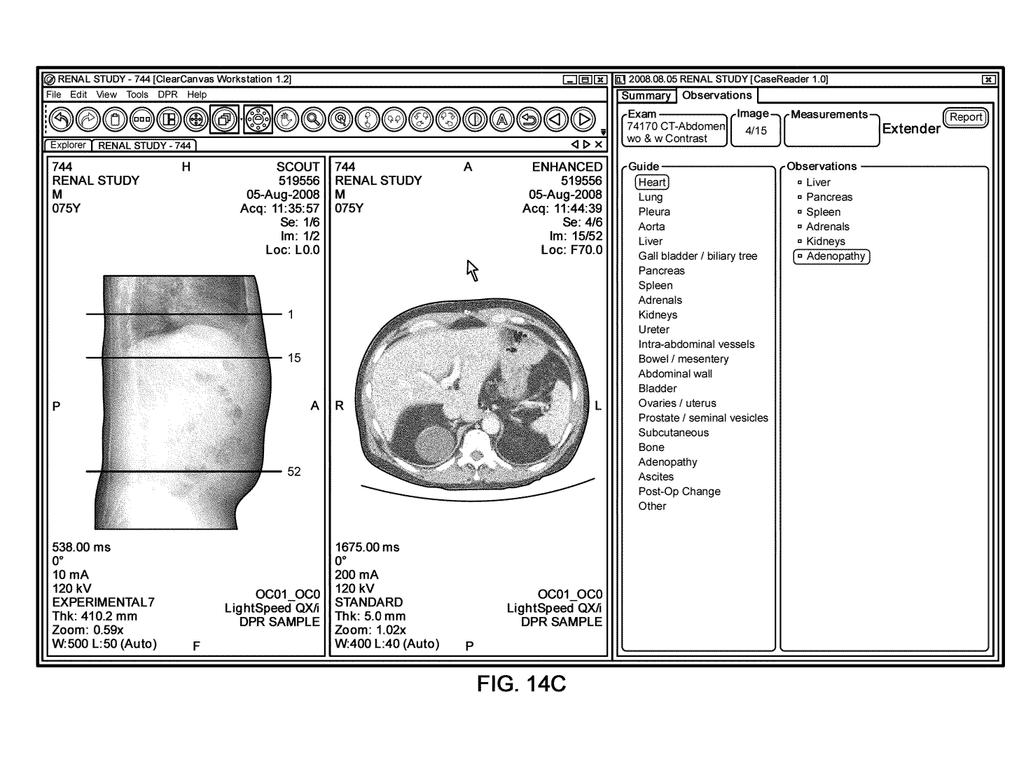

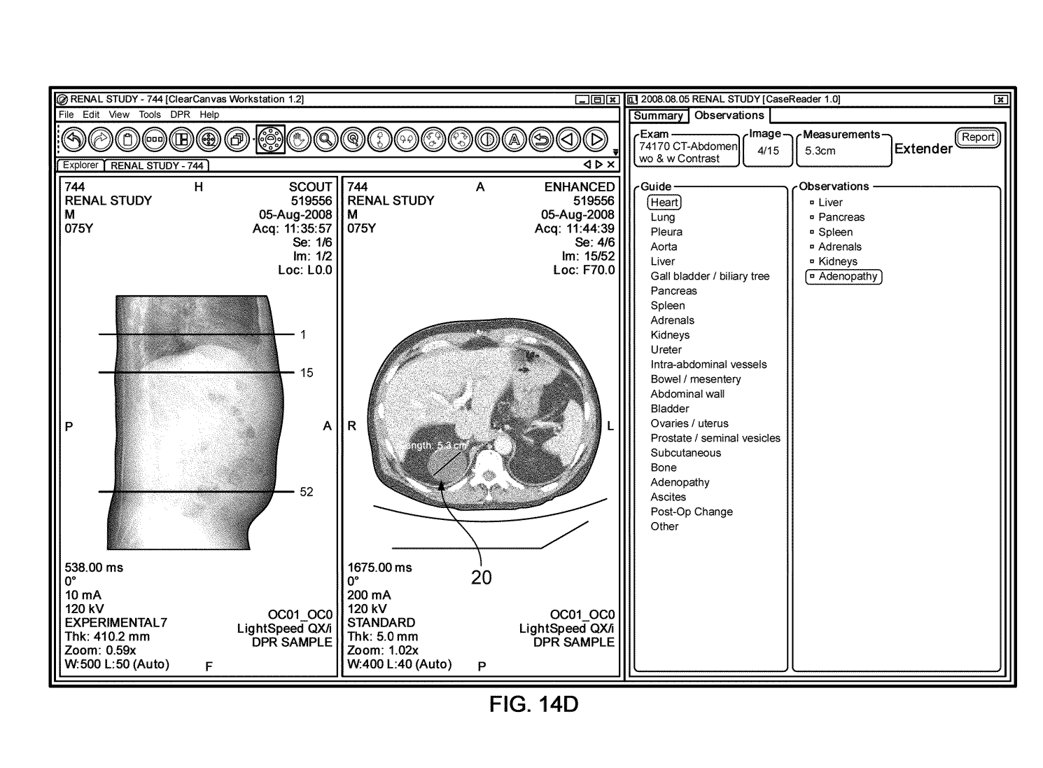

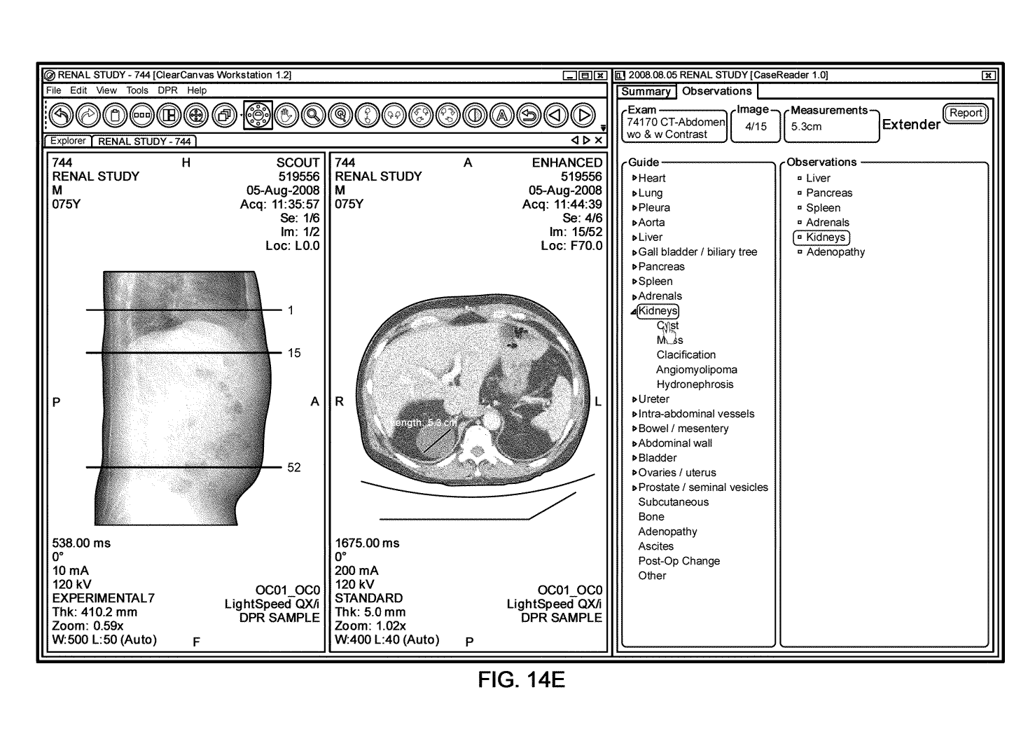

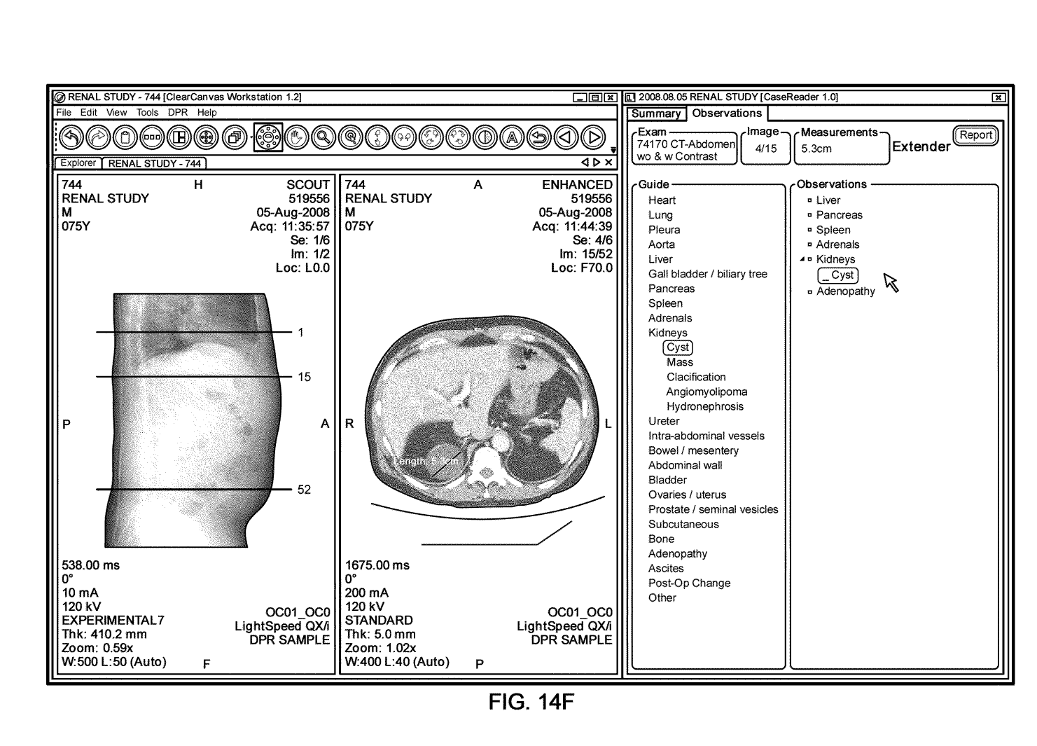

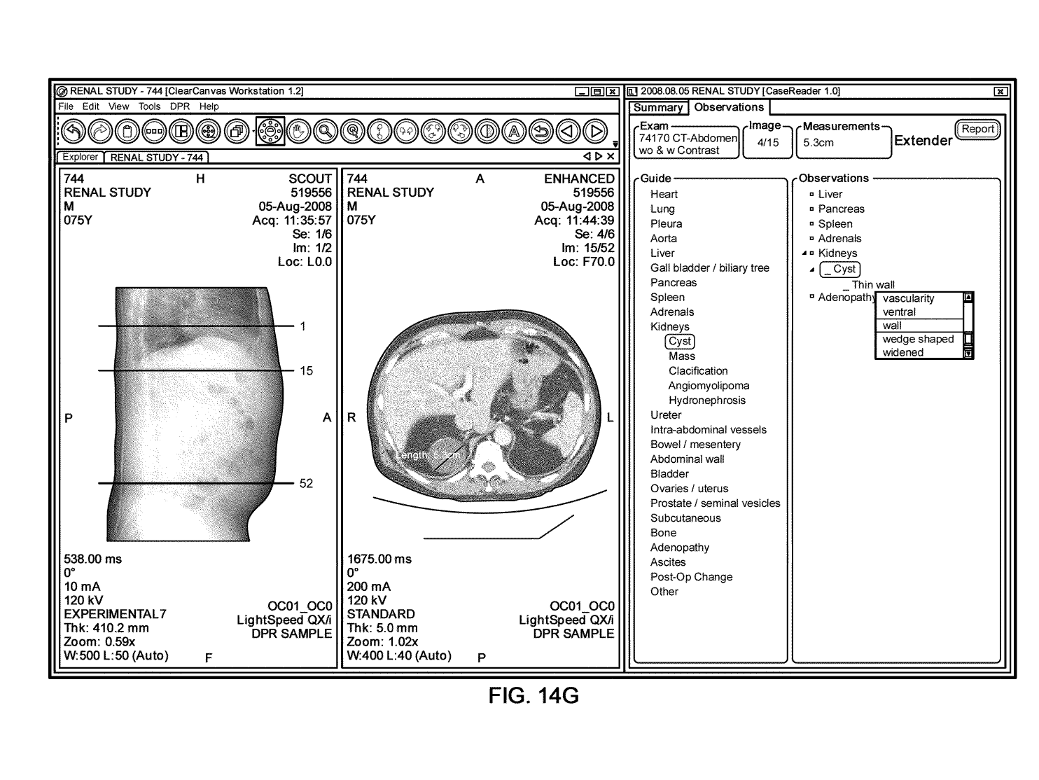

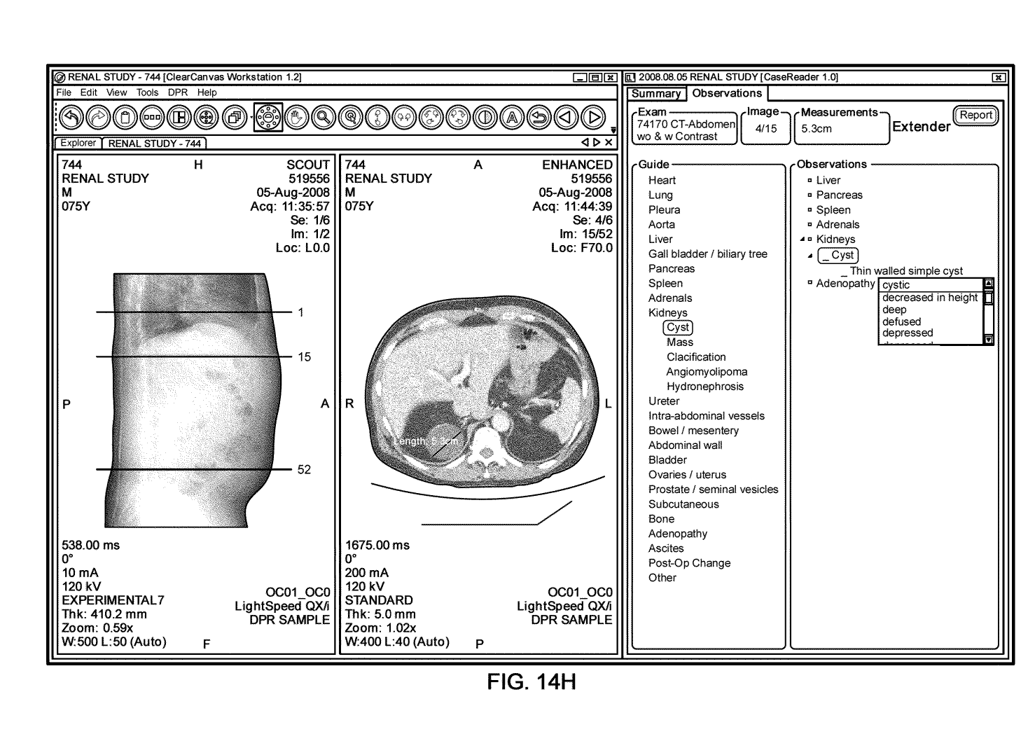

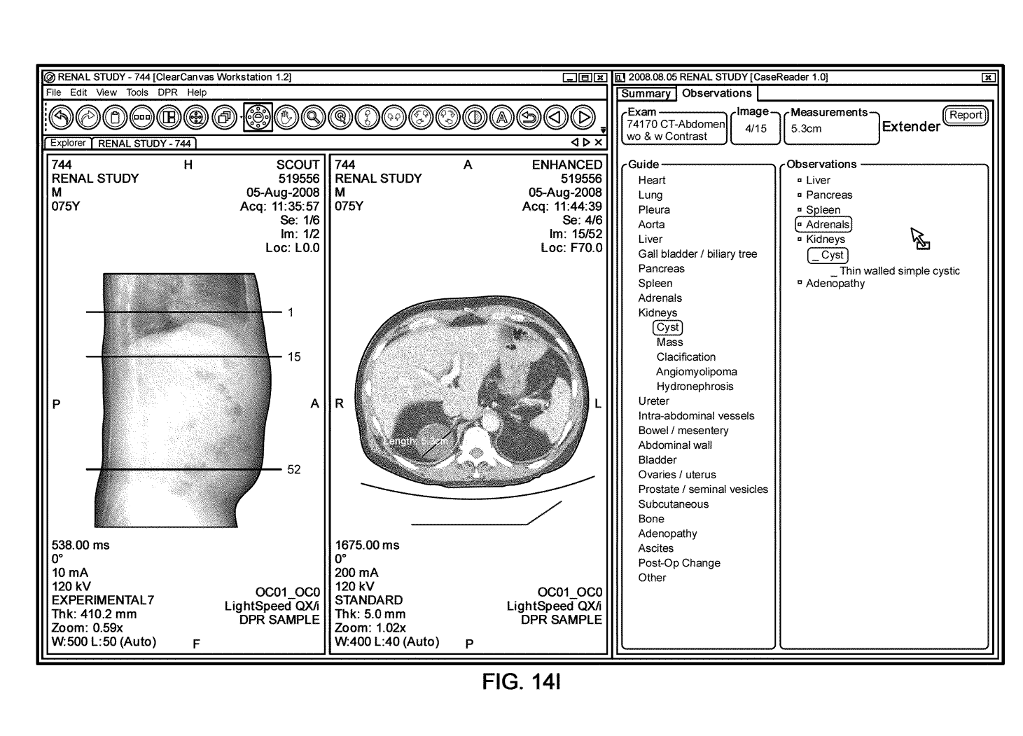

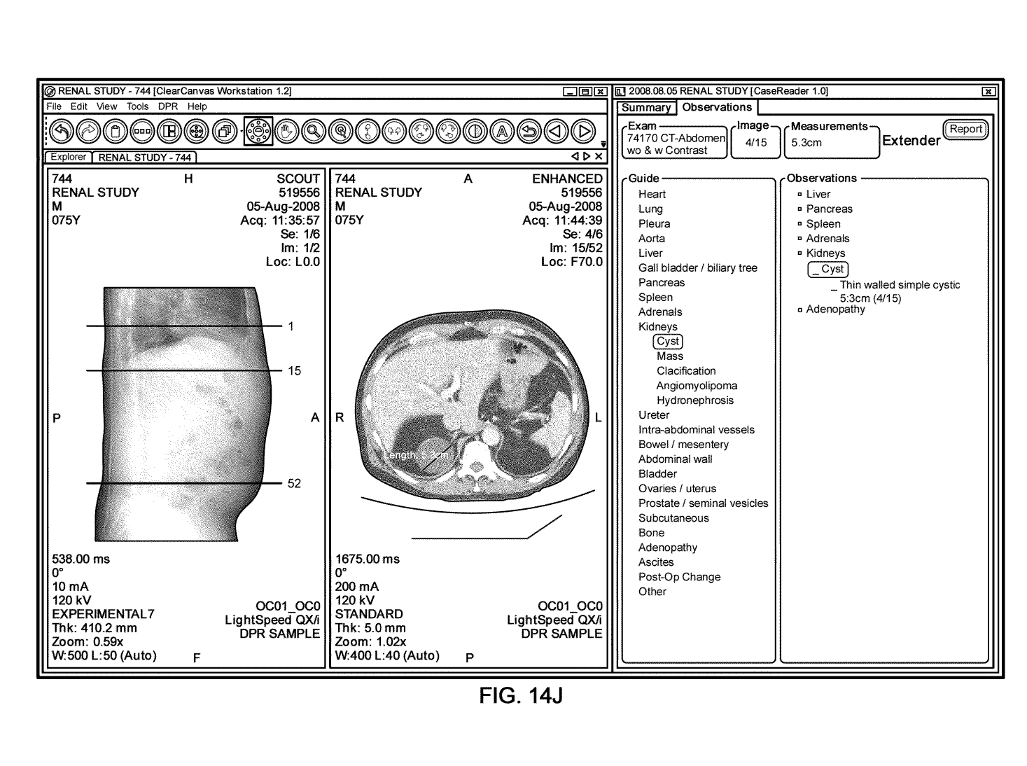

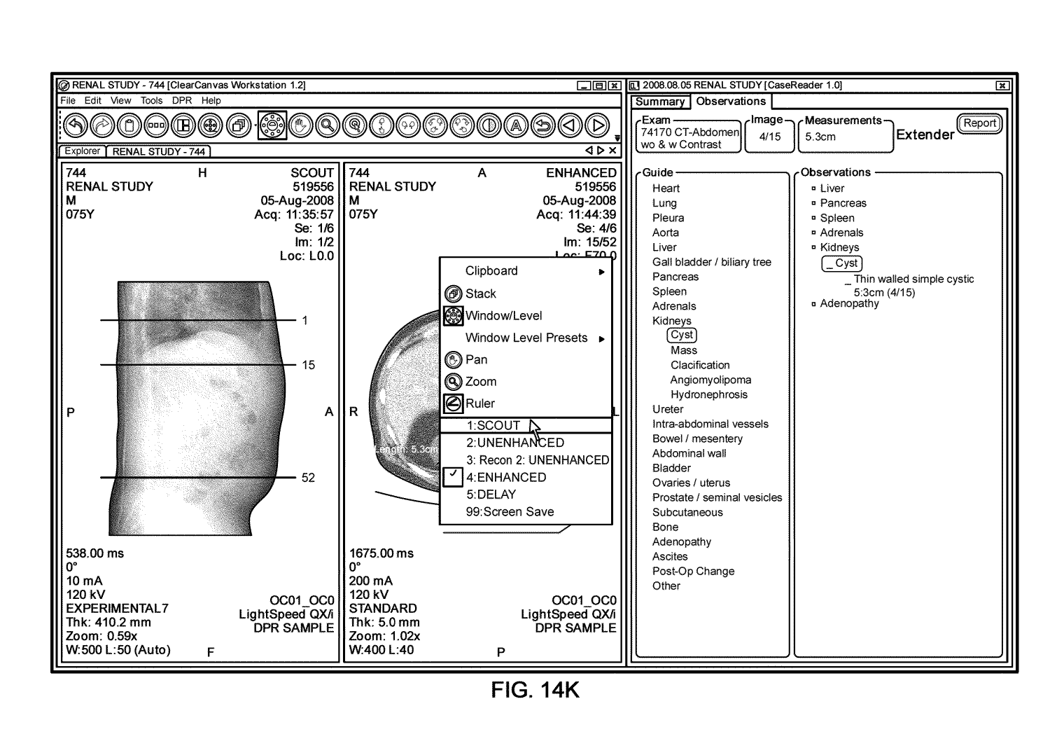

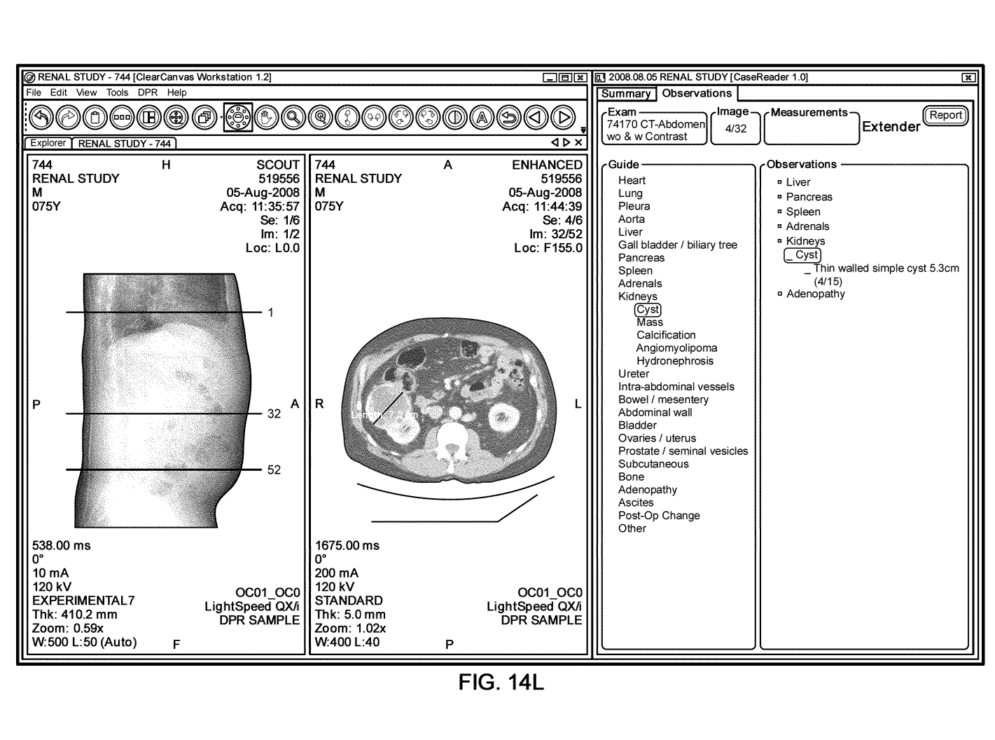

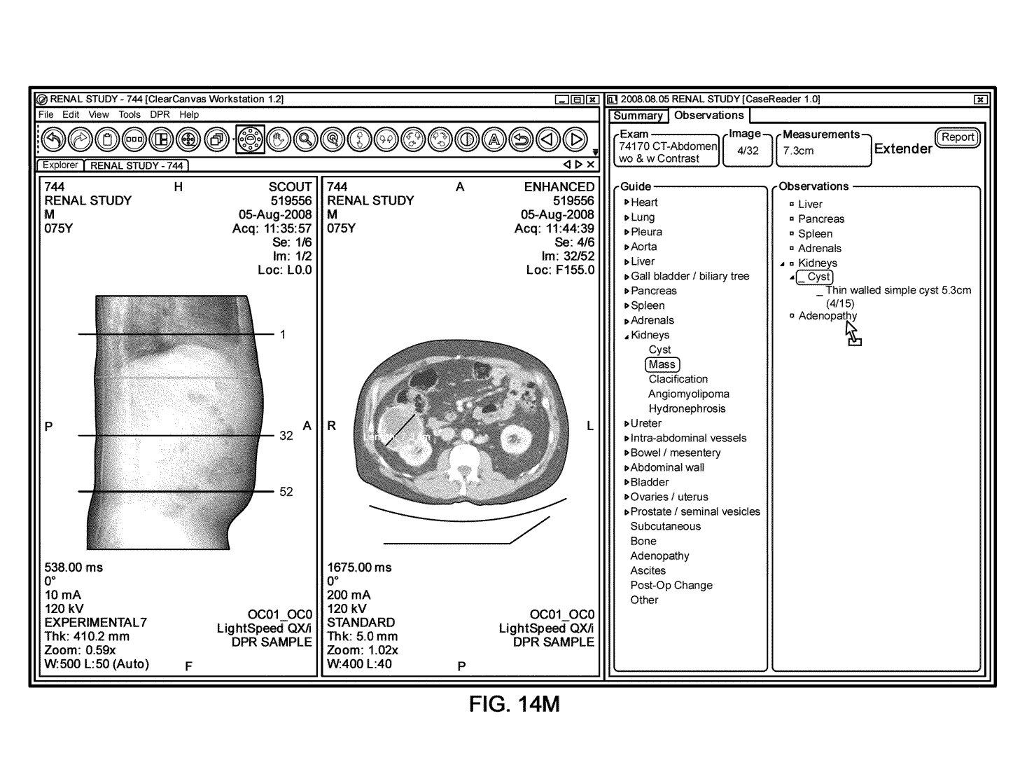

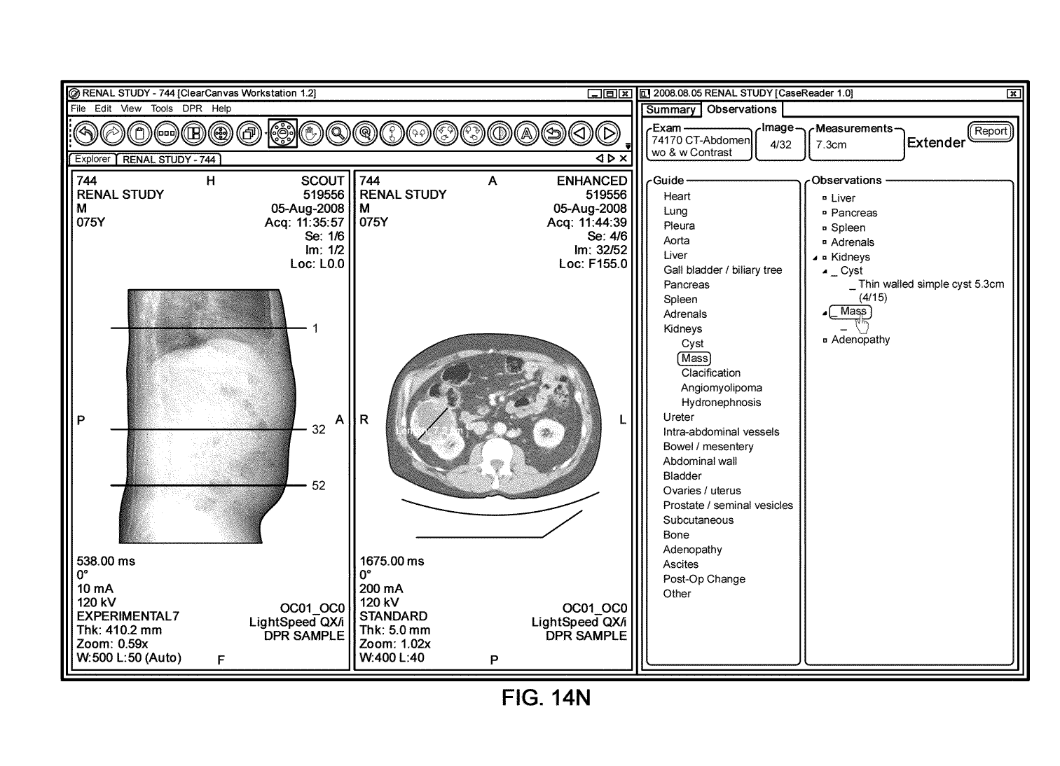

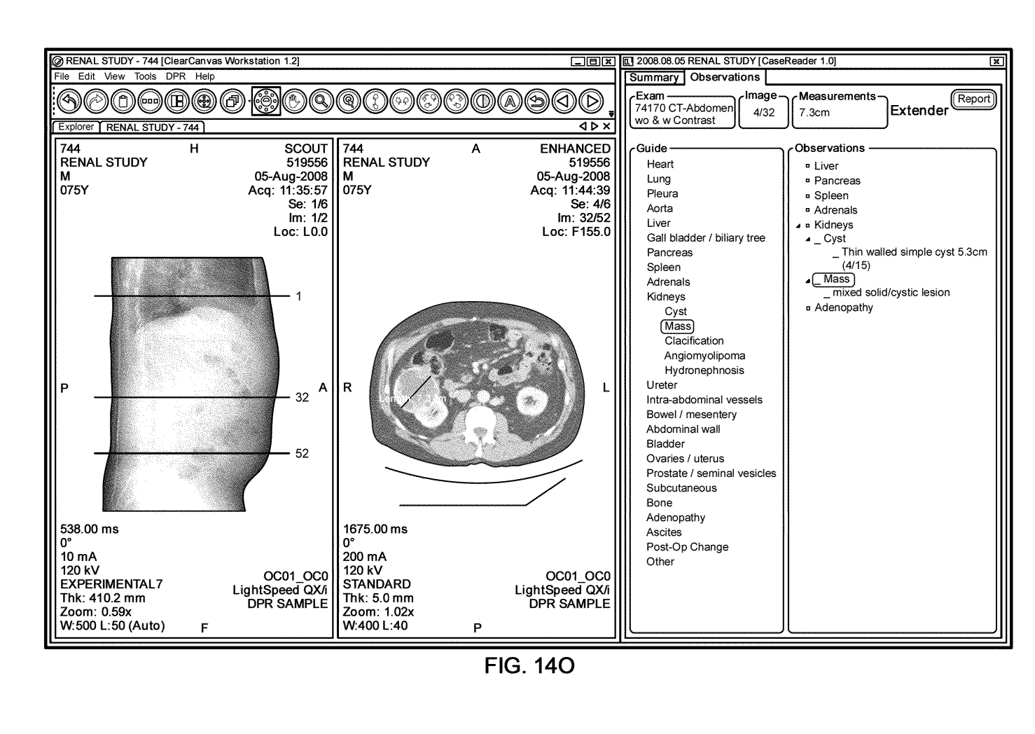

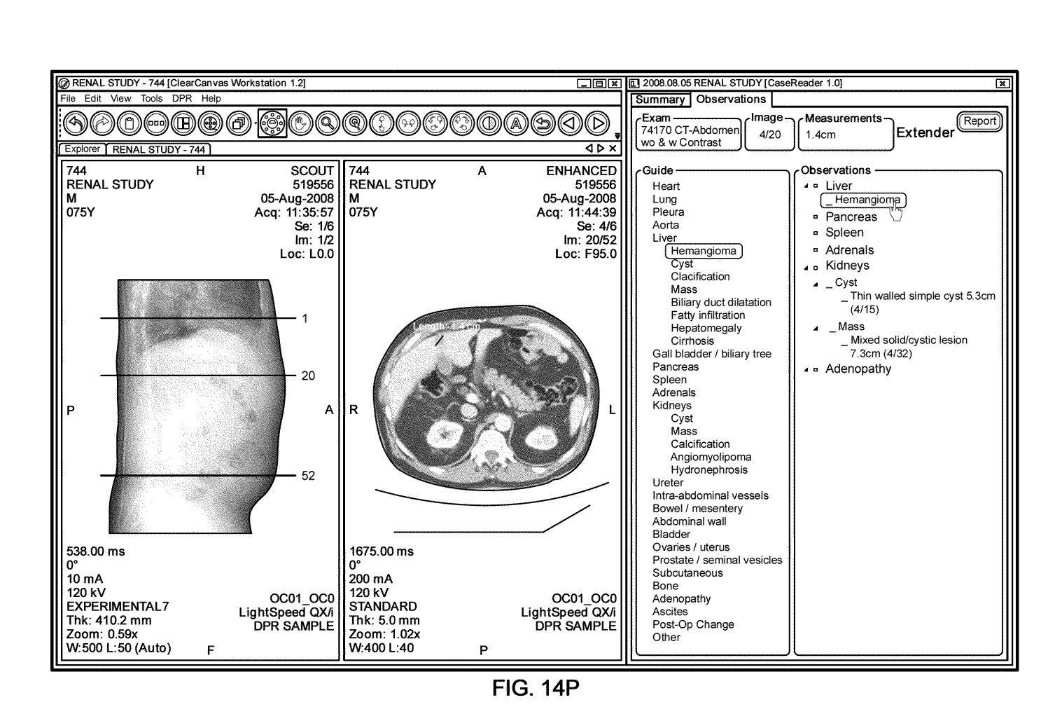

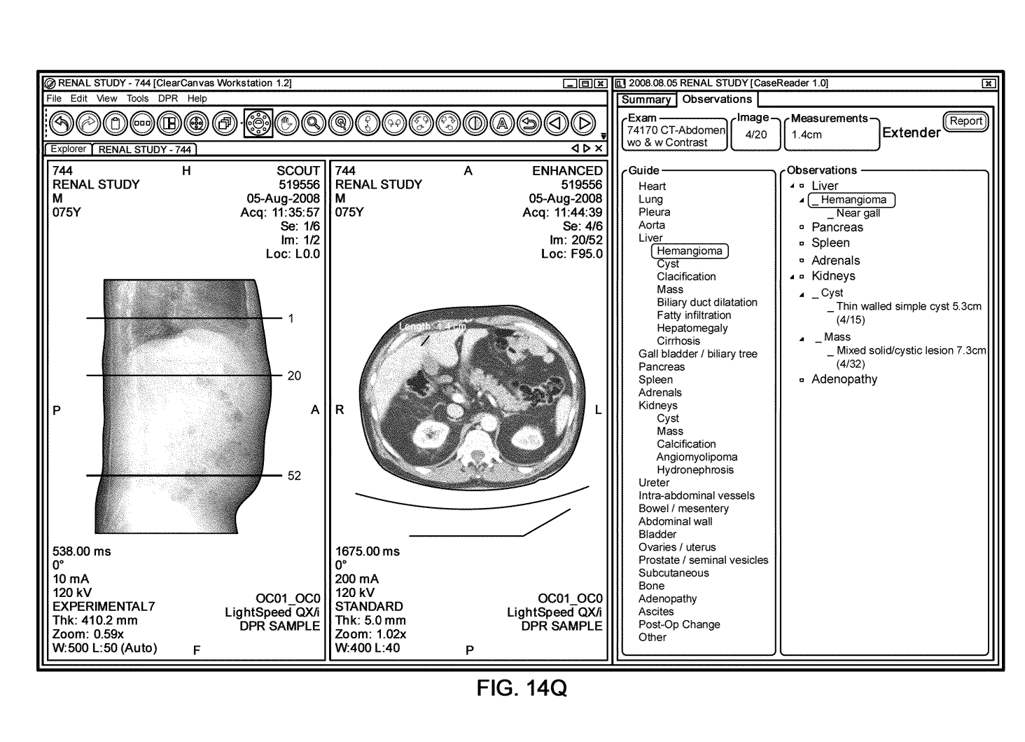

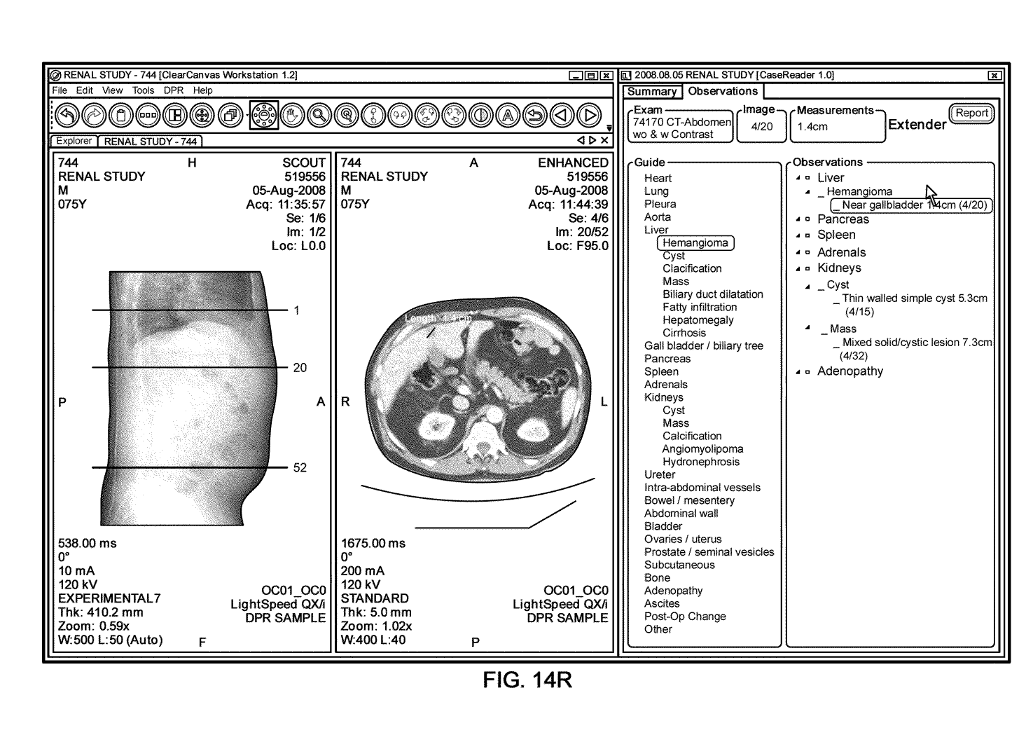

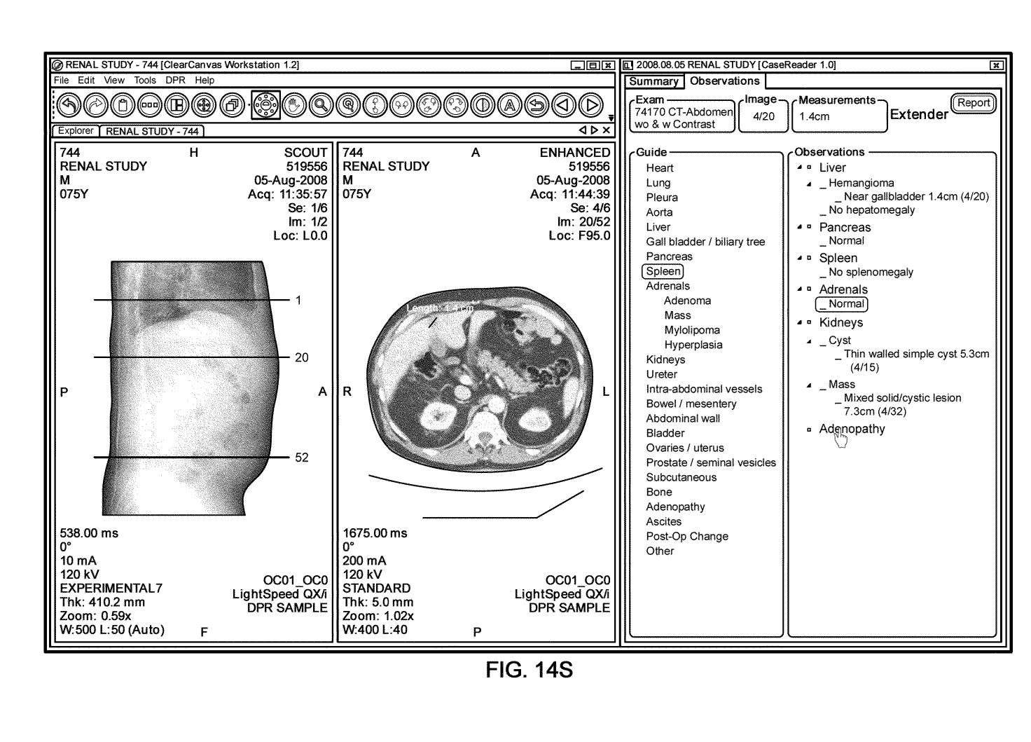

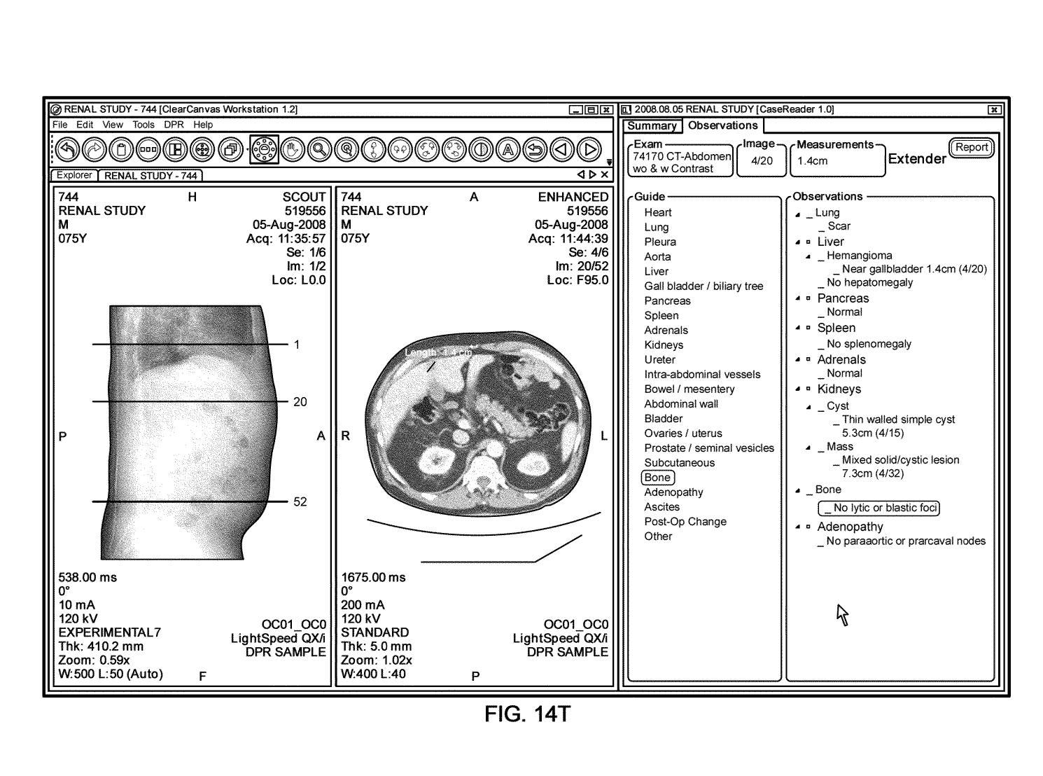

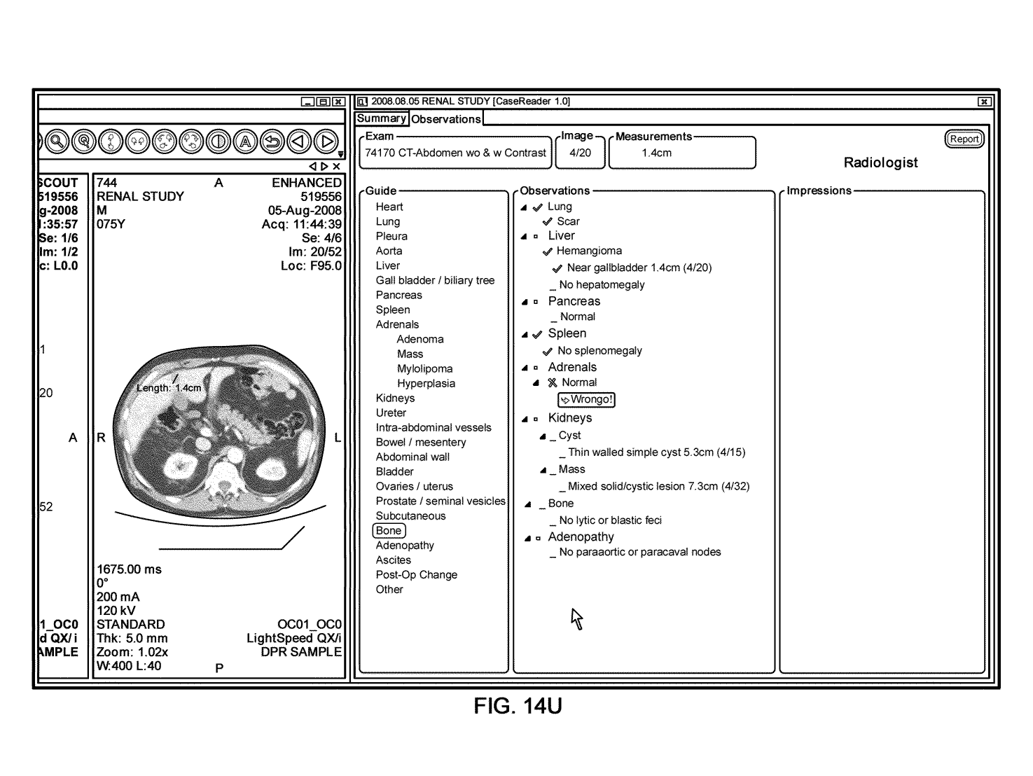

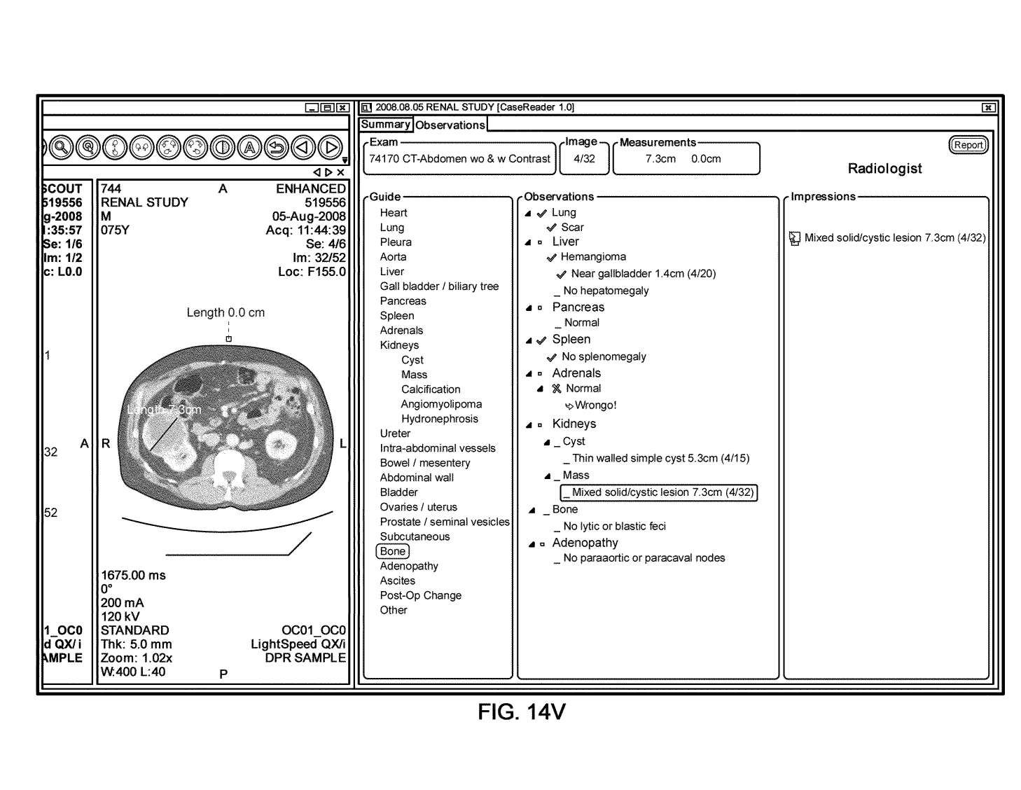

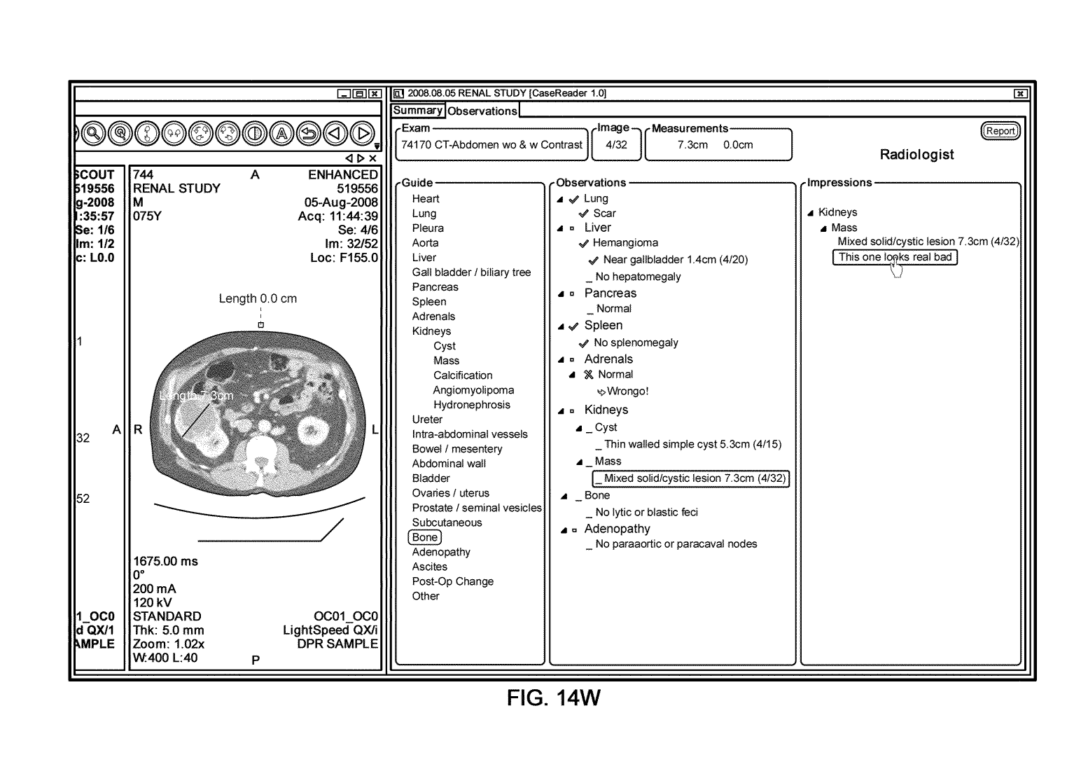

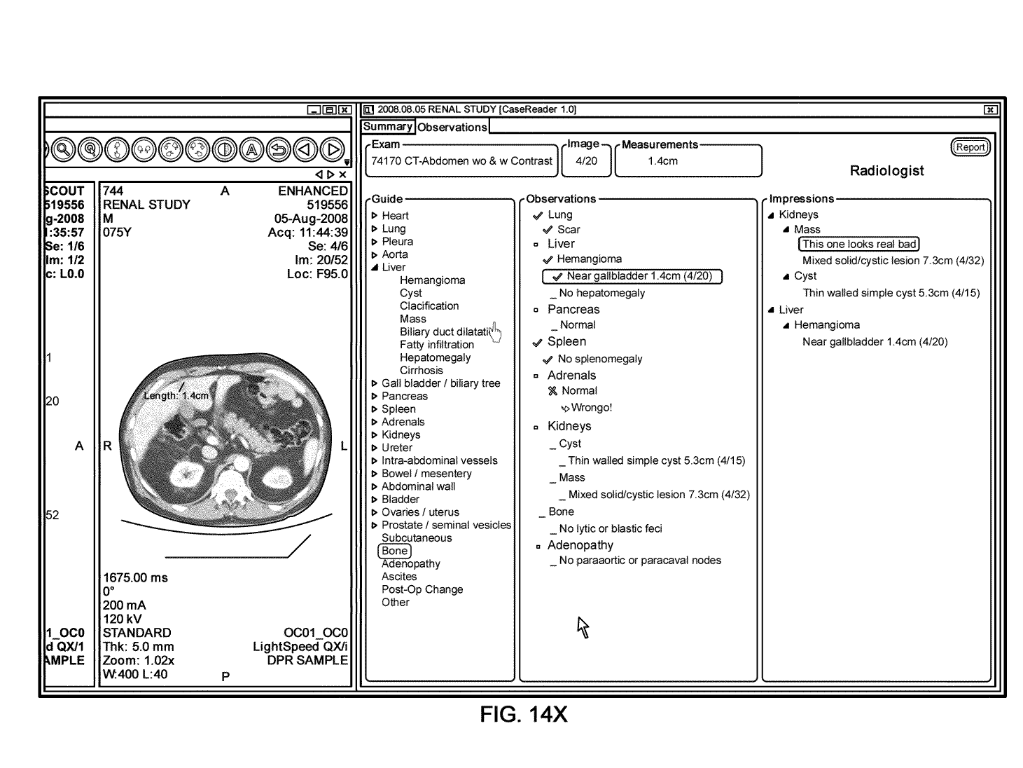

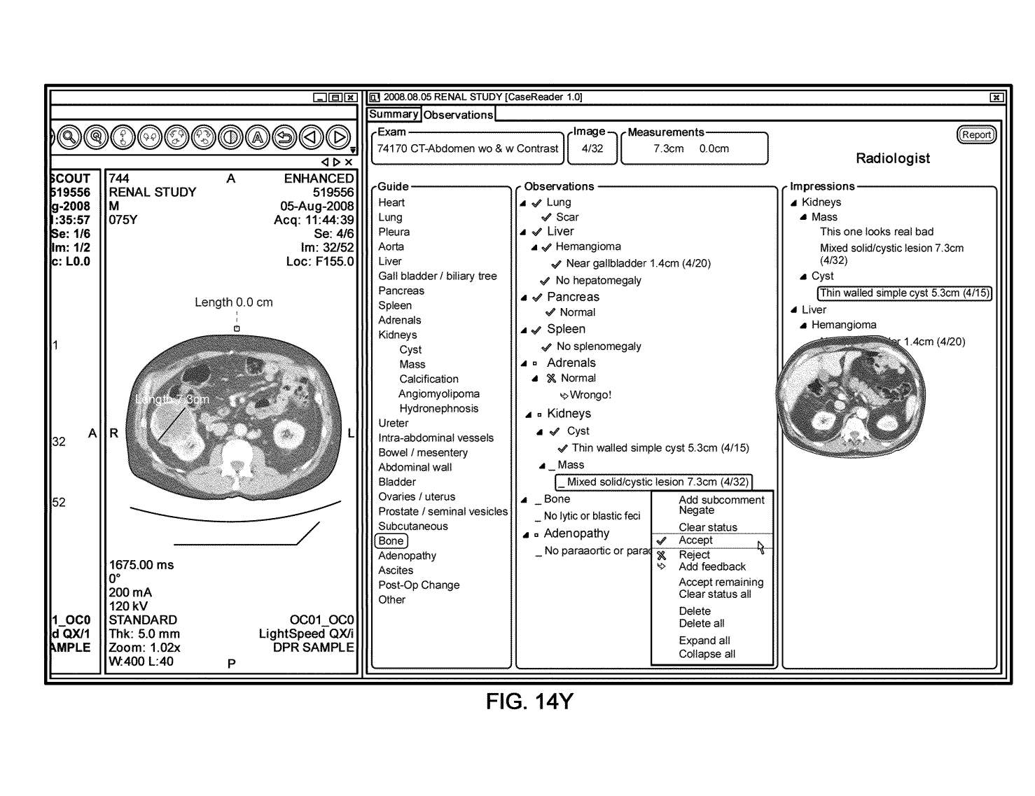

FIG. 14a illustrates that the window displaying the captured data (shown as two-dimensional data for illustrative purposes, but can be three-dimensional images instead or in addition to the two-dimensional images) can be presented with a window showing summary and observation tabs. The summary tab can display and edit the CPT code and title for the procedure, an indication being investigated, a patient history, prior exams, key image listings, two-dimensional data series lists, three-dimensional data series lists, the status of the analysis reporting by the diagnosticians, a log with optional dates and times when actions were taken with the study, and combinations thereof, as shown in FIG. 14a.

FIG. 14b illustrates that the Observations tab can list, for example in a Guide panel (as shown), all of the organ and/or segmentation groups, and/or the relevant organ and/or segmentation groups. Optionally or additionally, desired groups and organs can be dragged or otherwise manually or automatically selected and copied into an Observations panel. Notes can be automatically entered under each segmentation group (e.g., organ) in the Guide and/or Observations (as shown) panel, for example by the technician and/or automatically by the system based on a comparison with known data sets. The system can produce more structured and uniform reporting practices.

The Observations tab can also have a display showing which slice image or location is being observed (or to have the diagnostician enter a desired slice or location to retrieve), a measurements window that can show geometric measurements of mouse cursor movements over the images (e.g., with the mouse button held down, “dragging”, or automatic diameters measured when clicked on anatomical features).

FIG. 14b illustrates the Observations panel having segmentation groups with observations already included. FIG. 14c illustrates that the segmentation groups in the observations panel with no observations yet included.

FIG. 14d illustrates that the line can be drawn across abnormality 20. The line can be illustrated by the length (or any desired dimension, such as diameter) measurement of the line (e.g., “length 5.3 cm”, as shown). The length measurement of the line can appear in the measurement box of the Observations tab, as shown. The abnormality 20 can then be associated with one or more segmentation group and/or have its own segmentation group (e.g., adenopathy, as shown). For three-dimensional images (and two-dimensional views of adjacent scanned data), the system can calculate volumetric measurements for selected areas and/or segmentation groups.

FIG. 14e illustrates that the kidneys can be selected in the Observation panel. The Guide panel can produce a list of suggested observations for the desired segmentation group (in this example, the kidneys), such as a cyst, mass, calcification, angiomyolipoma and hydronephrosis. As shown in FIG. 14f, a suggested observation (e.g., cyst) can be selected by double-clicking or dragging the suggestion under the kidney inn the Observations panel.

FIGS. 14g through 14i illustrate that observations and additional details can be entered manually by the user (e.g., technician and/or radiologist). Suggested word choices can pop up, as shown, based on the typed word in-progress. Observations can be clustered together, such as for organizational purposes, for example by indentations.

FIG. 14j illustrates that the measurement can be dragged from the Measurement box in the Observation panel and/or from the image, and dropped into the text observation. The slice and/or other location indicator can also be automatically included or dragged and dropped into a specific line of the Observation.

FIG. 14k illustrates that the image set (e.g., scout, enhanced, unenhanced, etc.) can be selected and that the slice can be included with the given observation. When a report is later created, the selected slice image from the appropriate image set can be automatically included in the report.

FIGS. 14l through 14o illustrate that additional observations can be made regarding the same segmentation group in the same or a different slice or geometric location. For example, the kidneys can also be recorded as having a mass described as “mixed solid/cystic lesion 7.3 cm ( 4/32)” (i.e., 7.3 cm in diameter, in slice 4 of 32 slices).

FIGS. 14p through 14r illustrate that observations can be made regarding different segmentation groups in the same or different slices or geometric locations. For example, the liver can be recorded as having a hemangioma described as “near gallbladder 1.4 cm ( 4/20).”

FIG. 14s illustrates that segmentation groups can be labeled as “normal”, the adrenals as shown.

FIG. 14t illustrates yet another pathological observation in another segmentation group. For example, the bone can be recorded as “no lytic or blastic foci”, and other organs and segmentation groups can be labeled as desired.

When segmentation groups are required to be observed (e.g., for full reimbursement, and/or a standard of due care), the segmentation groups can be specially labeled (e.g., with an asterisk, as shown), and/or the observer can be required to complete the desired segmentation groups before a report can be produced.

FIG. 14u illustrates that when the initial observations are completed (e.g., by a technician), the file can be forwarded to a radiologist or other secondary diagnostician. The data from the initial observations, along with the text notes, dimensions, slice locations, and tagging of the images (e.g., for dimensioning or other desired marking) can be sent to the radiologist as well.

As shown in FIGS. 14u through 14y, a panel for the radiologist's impressions can allow the radiologist to keep his or her impressions distinct from the technician's observations. The radiologist can approve (indicated by a check mark, as shown in the pop-up menu in FIG. 14y) or reject (indicated by an “x”) the observations from the technician. Approved observations from the technician can be default copied into the radiologist's impressions panel if desired. The radiologist can manually drag and drop or double click to copy the technician's observations into the radiologist's impressions panel. The radiologist can use the suggest language in the Guide panel or pop-up box as available to the technician (or other diagnostician entering data in the observations panel). The radiologist can manually enter text, tag images and drag measurement and slice or location data into the impressions panel.

When the radiologist is satisfied with the data in the impressions panel, the radiologist can click the “report” button in the top right corner of the window. The system can then automatically generate a report.

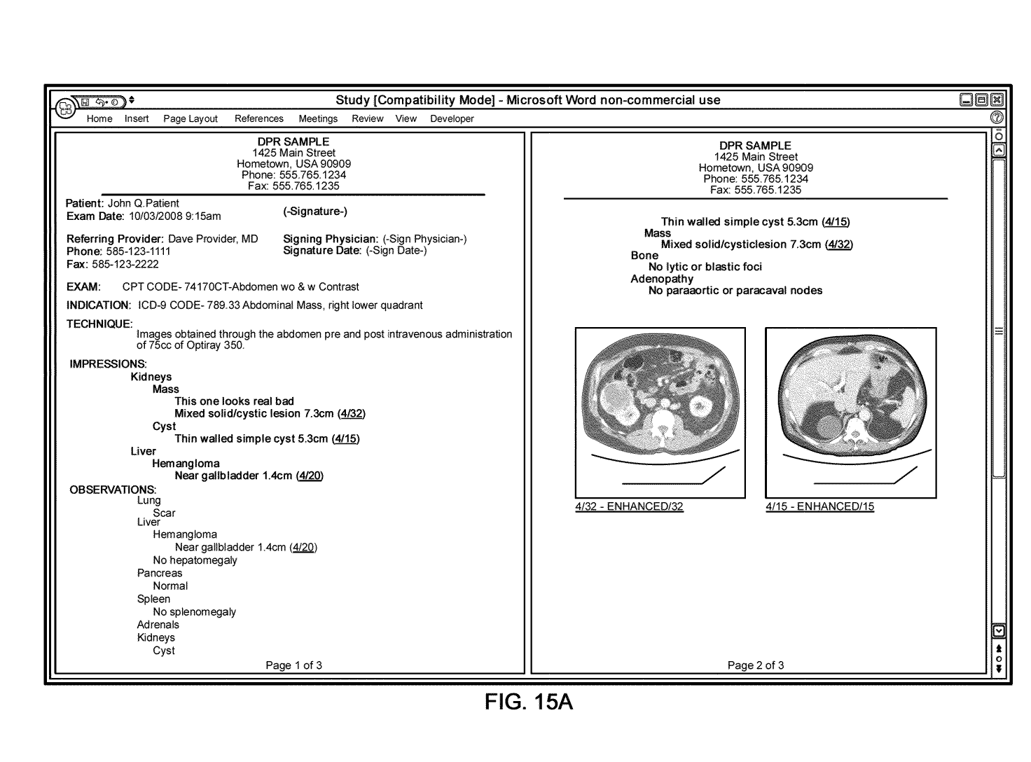

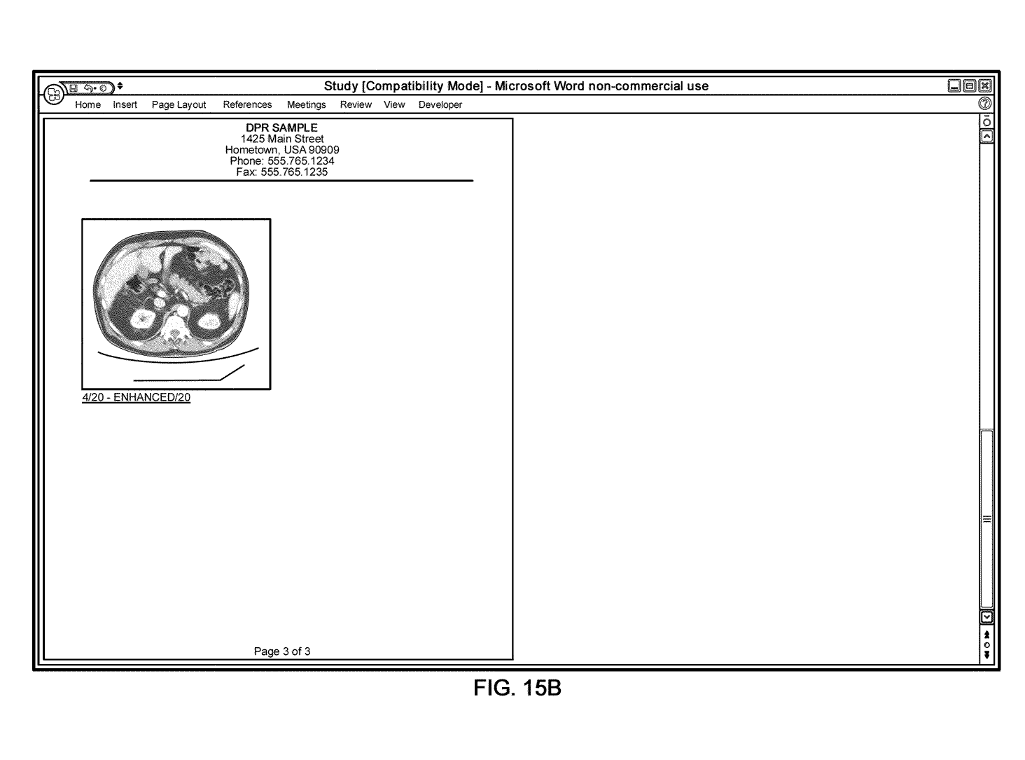

FIGS. 15a (e.g., pages 1 and 2) and 15b (e.g., page 3) illustrate that the system can automatically produce a complete report or report template from the data listed in the summary and observations tabs of the extender module. The report can be edited manually after creation. The report template can be edited so the system automatically creates a report with desired data and excludes undesired data. As shown, the report can include images tagged for inclusion in the report.

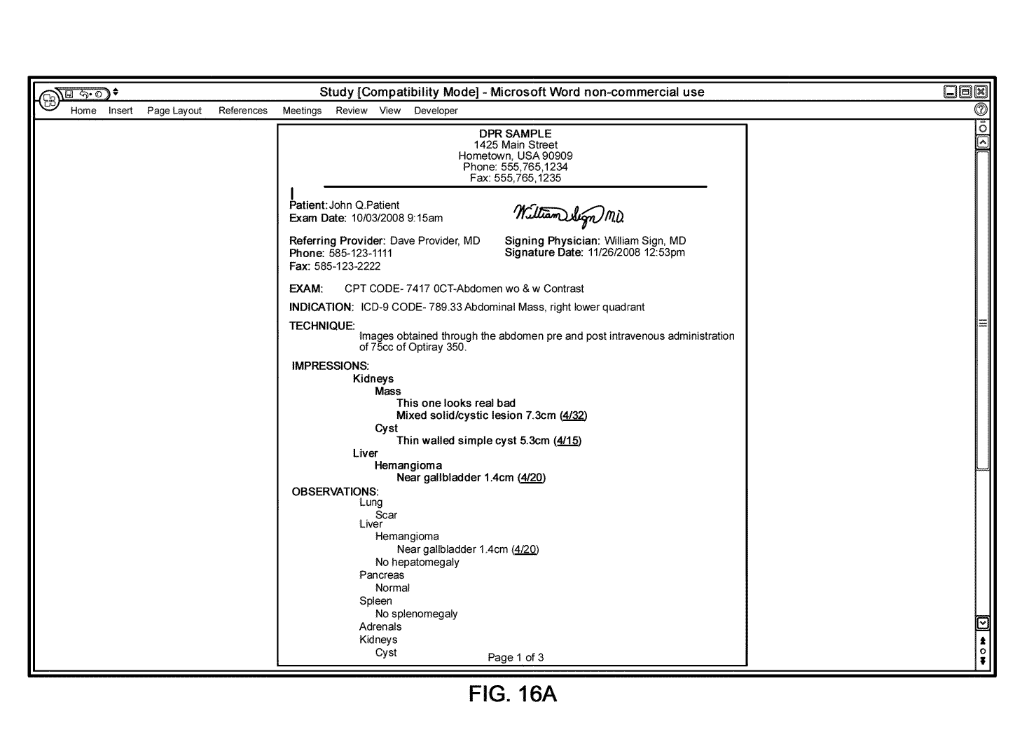

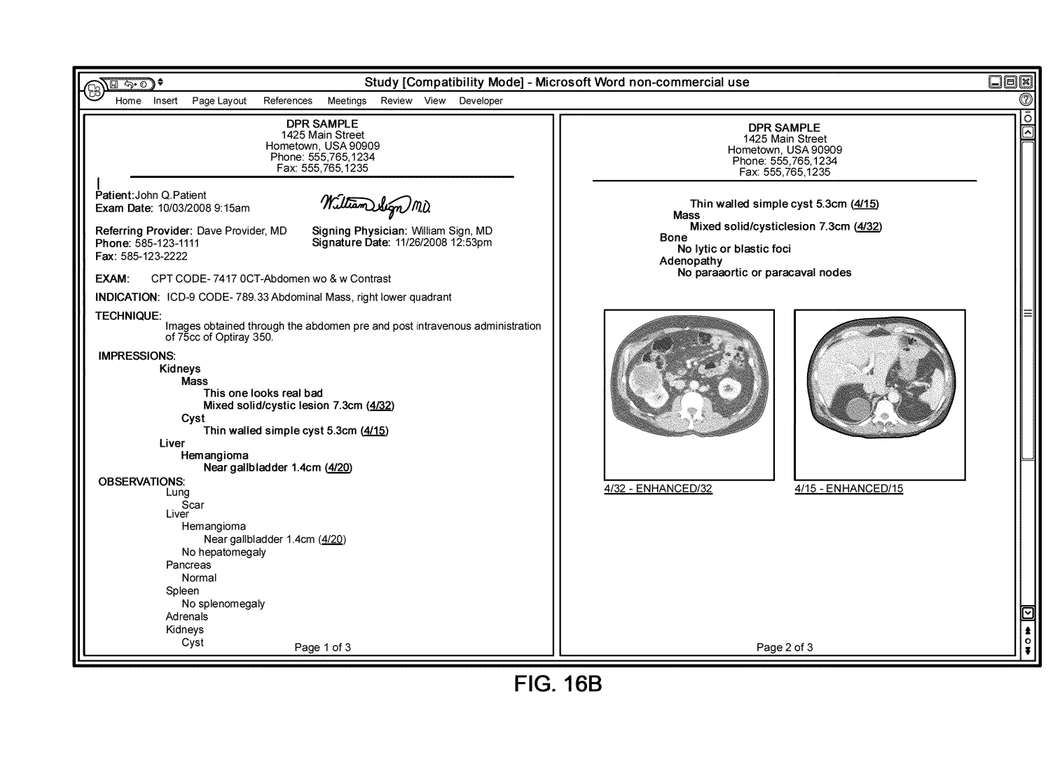

FIGS. 16a and 16b illustrate that once the report is approved, for example by the radiologist, a digital (as shown) or manual signature can be added before the report is sent to the desired recipients.

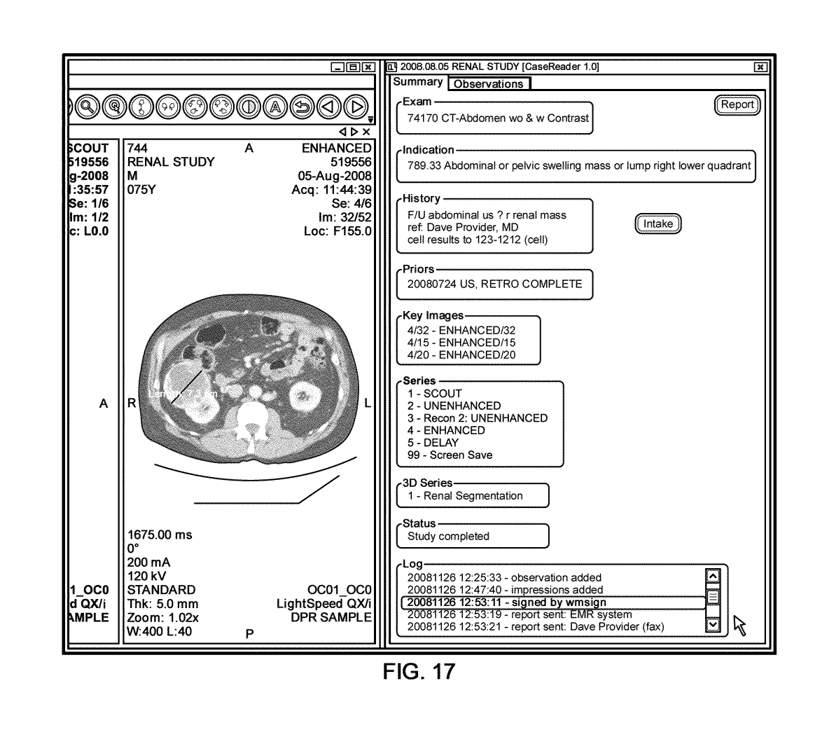

FIG. 17 illustrates that the system can automatically or manually enter actions performed into the log in the summary tab. The individual taking the action, time, date, location, or combinations thereof can be entered into the log with the action itself. For example, when the report is approved and signed by the physician, the system can automatically create a log entry that the report was approved and signed by the physician, along with the physician's name, date and time of approval, as shown. The log can be kept with the corpus of data.

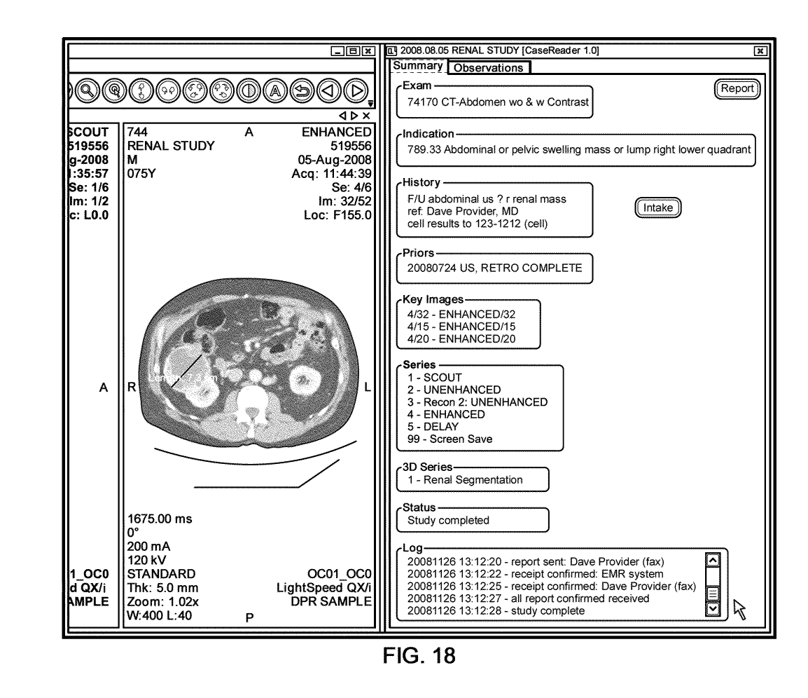

FIG. 18 illustrates that the system can automatically distribute electronic copies of the report to desired recipients, and enter delivery of the report into the log. For example, the system can send the report to the patient's insurance provider, primary care physician, and patient. The system can enter in the log when reports are confirmed received. The system can enter into the log when the study is complete. The system can close and lock the data for the study when the study is complete.

By using the disclosed system and methods for processing the patient study and data, the health care provider's “read” time expended per case can be significantly reduced. The health care provider's time per case might be reduced from about 15 to 20 minutes (typical time now) to just less than 5 minutes. Normal exams will take a lot less time to read as well.

The system and method disclosed herein can be used for teleradiology or local radiology. Teleradiologists can use the system and methods at data centers and/or remote locations (e.g., even internationally). The system and methods can be used for patients to receive and review their own data sets.

The system and methods disclosed herein can be used on or with a remote computing device, such as a portable device, such as a PDA or cellular phone. For example, the organ or segmentation data of interest alone can be transmitted to the remote device in lieu of the entire data set or selected slkes of data.

The system can be linked to a PACS system, for example for analytical purposes to filter criteria based on image sets. For example, the system can search for all volumetric masses of 1.7 cm or larger in the kidney (or other size or anatomical location) within the library of data sets.

The terms software function and software module are used interchangeably herein. The term health care provider can include the radiologist, a cardiologist, physician's assistant, other health care professional, or combinations thereof.

The system can be used at local, outpatient, remote viewing locations, or combinations thereof. The system can be used for diagnostic radiology (CAD is a technology tool used for diagnostic radiology). The system can have a module to convert output between various languages (e.g., English, Spanish, French, Mandarin, Cantonese, etc.), for example, for the anatomical library and the GUI.

As used herein, screen shot is synonymous with screen capture and anatomical feature is used interchangeably with segmentation group.

It is apparent to one skilled in the art that various changes and modifications can be made and equivalents employed without departing from the scope of the invention disclosed. Elements shown with the specific variations shown herein are exemplary for the specific variation shown and can be used in combination with the other variations and other elements shown herein.

We claim:

1. A method for processing medical imaging data by a computer system, the method comprising:
acquiring a first corpus data set comprising a three-dimensional data set;
comparing reference database data to the three-dimensional data set;
automatically segmenting the three-dimensional data set, wherein segmenting comprises identifying and labeling anatomical features within the three-dimensional data set;
navigating through the three-dimensional data set;
annotating the three-dimensional data set;
generating an electronic file comprising inserting patient data in an electronic template and comprising automated detection diagnosing;
altering the electronic file comprising adding observations to the electronic file after generating the electronic file; and
transmitting the electronic file from a first computer system to a second computer system.

2. The method of claim 1, wherein comparing further comprises scaling the acquired first corpus data with respect to the reference database data.

3. The method of claim 2, wherein scaling comprises deforming processing more than one distinct deformation vector to deform the acquired first corpus data.

4. The method of claim 3, further comprising comparing the first corpus data set to a past corpus data set for the same patient.

5. The method of claim 4, further comprising tagging and/or highlighting data in the first corpus data set, wherein highlighting comprises comparing with the past corpus data set.

6. The method of claim 1, further comprising altering the opacity of a feature in the three-dimensional data set.

7. The method of claim 1, further comprising navigating through the two-dimensional data set.

8. The method of claim 1, further comprising the first computer transmitting the electronic file to a third computer over a network, wherein the electronic file comprises data edited for a user of the third computer.

9. The method of claim 1, wherein annotating comprises linking observational text data to the data set.

10. The method of claim 1, wherein generating the electronic file comprises executing an algorithm to increase diagnostic efficiency.

11. A method for displaying medical corpus data, the method comprising:
visually displaying at least one two-dimensional image concurrent with a three-dimensional volumetric image corresponding to the at least one two-dimensional image; and
visually displaying synchronous three-dimensional and two-dimensional navigation through the at least one two-dimensional image and the three-dimensional volumetric image.

12. The method of claim 11, further comprising:
acquiring a first set of three-dimensional data points, constructing the at least one two-dimensional image from at least some of the three-dimensional data points; and
constructing the three-dimensional volumetric image from at least some of the three-dimensional data points.

13. The method of claim 11, further comprising measuring a geometric measurement of a feature in the first three-dimensional volumetric image, and creating an electronic file comprising the measurement.

14. The method of claim 13, further comprising measuring a geometric measurement of the feature in one of the at least one two-dimensional images, and synchronously displaying the geometric dimension in the first three-dimensional volumetric image.

15. A method of diagnosing a patient using a first computer system comprising a radiological corpus data set, the method comprising:
segmenting the corpus data set based on tissue-specific parameters, wherein the segmented data set comprises tissue-identifying data; wherein segmenting comprises automatically identifying a seed voxel in an anatomical feature in the data set, and linking a label of the anatomical feature to the location in the data set of the anatomical feature.

16. The method of claim 15, further comprising adjusting the transparency of the segmented data based on the tissue-identifying data.

17. The method of claim 15, wherein the tissue-identifying data comprises groups of organs.

18. The method of claim 15, wherein the corpus data comprises three-dimensional data, and wherein the method further comprises two-dimensional sectioning of the three-dimensional data.

19. The method of claim 15, further comprising logging within the data set actions taken with the data set.

20. The method of claim 15, wherein the corpus data set comprises a three-dimensional data set, and further comprising constructing a three-dimensional volume from the corpus data set, wherein the three-dimensional volume comprises voxels, and wherein the labeling comprises associating at least one of the voxels with the anatomical labels.
